(19) United States
(12) Patent Application Publication
(10) Pub. No.: US 2024/0230666 A1
(43) Pub. Date: Jul. 11, 2024

(54) COMPOSITIONS AND METHODS FOR DETECTING, PREVENTING, AND TREATING DISTURBED MICROBIOTA-IMMUNE HOMEOSTASIS

(71) Applicants: THE REGENTS OF THE UNIVERSITY OF MICHIGAN, Ann Arbor, MI (US); INSTITUTE FOR SYSTEMS BIOLOGY, Seattle, WA (US)

(72) Inventors: Helmut Grasberger, Ann Arbor, MI (US); John Y. Kao, Ann Arbor, MI (US); Andrew Magis, Seattle, WA (US)

(21) Appl. No.: 18/284,033

(22) PCT Filed: Mar. 24, 2022
(86) PCT No.: PCT/US22/21701

§ 371 Date: Sep. 25, 2023

Related U.S. Application Data
(60) Provisional application No. 63/166,078, filed on Mar. 25, 2021.

(51) Int. Cl. G01N 33/68; C12Q 1/6883; C12Q 1/689; G01N 33/573; C12Q 1/6874

(52) U.S. Cl. CPC G01N 33/6863; C12Q 1/6883; C12Q 1/689; G01N 33/573; C12Q 1/6874; C12Q 2600/156; C12Q 2600/158; G01N 2333/54; G01N 2800/065; G01N 2800/52

(57) ABSTRACT

The present invention relates to methods for detecting disease-relevant microbial colonization of the gut mucosal surface (proinflammatory mucosal dysbiosis) prior to onset of overt inflammation by measuring a level of interleukin 17C (IL17C) and other microinflammation markers in a biological sample from a subject and treating and/or preventing intestinal inflammation if these markers are elevated.

### CROSS REFERENCE TO RELATED APPLICATIONS

This application claims benefit of priority to U.S. Provisional Application No. 63/166,078, filed Mar. 25, 2021, the contents of which are incorporated herein by reference in their entirety.

### STATEMENT REGARDING FEDERALLY SPONSORED RESEARCH OR DEVELOPMENT

This invention was made with government support under DK117565 awarded by the National Institutes of Health. The government has certain rights in the invention.

### FIELD OF THE INVENTION

The present invention relates to methods for detecting disease-relevant microbial colonization of the gut mucosal surface (proinflammatory mucosal dysbiosis) prior to onset of overt inflammation by measuring a level of interleukin 17C (IL17C) and other microinflammation markers in a biological sample from a subject and treating and/or preventing intestinal inflammation if these markers are elevated.

### INTRODUCTION

The microbiome plays an important role in maintaining physiological functions of the body, and dysbiosis of the microbiome can lead to various disorders (e.g., intestinal inflammation).

Improved methods for detecting, treating, ameliorating, and preventing dysbiosis of the microbiome are needed.

### SUMMARY OF THE INVENTION

The present invention relates to methods for detecting mucosal dysbiosis through measuring a level of IL17C in a biological sample from a subject, as well as intestinal (micro-/macro-)inflammation through measuring a level of inflammatory markers (IL17A, IL6, CCL20, CXCL9, CCL11, CXCL11, FGF23, CRP, SAA1, and S100A8) and treating and/or preventing intestinal inflammation through, if elevated, treating and/or preventing proinflammatory dysbiosis.

In certain embodiments, the present invention provides a method, comprising:
measuring an IL17C level in a biological sample obtained from a subject;
characterizing the measured IL17C level within an established IL17C range;
measuring the levels of one or more of interleukin 17A (IL17A), interleukin 6 (IL6), C—C motif chemokine ligand 20 (CCL20), C—X—C motif chemokine ligand 9 (CXCL9), C—C Motif Chemokine Ligand 11 (CCL11), C—X—C motif chemokine ligand 11 (CXCL11), Fibroblast growth factor-23 (FGF23), C-reactive protein (CRP), serum amyloid A (SAA1), and neutrophilic marker calprotectin (S100A8) within the biological sample if the measured IL17C level is characterized as elevated within the established IL17C range;
characterizing the one or more measured IL17A, IL6, CCL20, CXCL9, CCL11, CXCL11, FGF23, CRP, and S100A8 levels within established ranges for IL17A, IL6, CCL20, CXCL9, CCL11, CXCL11, FGF23, CRP, SAA1, and S100A8 levels;
characterizing an intestinal inflammation status for the subject based upon the characterized one or more IL17C, IL17A, IL6, CCL20, CXCL9, CCL11, CXCL11, FGF23, CRP, SAA1, and S100A8 levels; and
treating the characterized intestinal inflammation status in the subject.

Such methods are not limited to a particular type of subject. In some embodiments, the subject is a human subject suffering or at risk of suffering from a breakdown of microbiota/immune system homeostasis. In some embodiments, the subject is a human subject suffering or at risk of suffering from an expansion of proteobacteria pathobionts. In some embodiments, the subject is a human subject suffering or at risk of suffering from inflammatory bowel disease (IBD) due to a loss of microbiota/immune system homeostasis at gut epithelial surfaces. In some embodiments, the subject is a human subject who has IBD, is diagnosed with IBD, is suspected to have IBD, is likely to have IBD, has one or more signs or symptoms of IBD (e.g., gastrointestinal, systemic, and extraintestinal symptoms), has increased risk for developing IBD based on positive family history or the presence of one or more risk variants in IBD susceptibility genes. In some embodiments, the subject is a human subject who has been previously diagnosed with irritable bowel syndrome (IBS), obesity, metabolic syndrome, hepatic encephalopathy, or colon cancer.

Such methods are not limited to a particular type or kind of biological sample. In some embodiments, the biological sample is a blood sample (e.g., plasma, serum, whole blood). In some embodiments, the biological sample is a tissue sample (e.g., an intestinal tissue sample).

Such methods are not limited to a particular type or kind of established marker (e.g., wherein the marker is one of IL17C, IL17A, IL6, CCL20, CXCL9, CCL11, CXCL11, FGF23, CRP, SAA1, and S100A8). In some embodiments, the established marker range is an established range of levels for that specific marker generated from a plurality of subjects (e.g., human subjects) (e.g., human subjects not suffering from intestinal inflammation and human subjects suffering from intestinal inflammation). In some embodiments, a measured IL17C level characterized as elevated is within the top 10% of the established 1L17C level range. In some embodiments, a measured IL17C level characterized as elevated is within the top 5% of the established 1L17C level range. In some embodiments, a measured IL17C level characterized as elevated is within the top 2% of the established 1L17C level range. In some embodiments, a measured IL17C level characterized as elevated is within the top 1% of the established 1L17C level range.

In some embodiments, the subject is characterized as not having mucosal dysbiosis if the measured levels of IL17C are characterized as not elevated in comparison with the established IL17C level. In some embodiments, the subject is characterized as having mucosal dysbiosis without loss of homeostasis (LOH) if the measured level of IL17C is characterized as elevated within the established IL17C level range, and each of IL17A, IL6, CXCL9, CCL11, CXCL11, FGF23, CRP, SAA1, and S100A8 is characterized as not elevated within the established range of levels for each specific marker. In some embodiments, the subject is treated through the administration of a therapeutically effective amount of one or more agents selected from a prebiotic agent, a probiotic agent, and a postbiotic agent.

In some embodiments, the prebiotic agent is selected from the group consisting of: complex carbohydrates, complex sugars, resistant dextrins, resistant starch, amino acids, peptides, nutritional compounds, biotin, polydextrose, fructooligosaccharide (FOS), galactooligosaccharides (GOS), inulin, starch, lignin, psyllium, chitin, chitosan, gums (e.g. guar gum), high amylose cornstarch (HAS), cellulose, s-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, xylooligosaccharides (XOS), locust bean gum, β-glucans, methylcellulose, and any combination thereof.

In some embodiments, the prebiotic agent is an oligosaccharide.

In some embodiments, the prebiotic agent is inulin.

In some embodiments, the prebiotic agent is selected from the group consisting of: amino acids, ammonium nitrate, amylose, barley mulch, biotin, carbonate, cellulose, chitin, choline, fructooligosaccharides (FOSs), fructose, galactooligosaccharides (GOSs), glucose, glycerol, heteropolysaccharide, histidine, homopolysaccharide, hydroxyapatite, inulin, isomaltulose, lactose, lactulose, maltodextrins, maltose, mannooligosaccharides, tagatose, nitrogen, oligodextrose, oligofructoses, oligofructose-enriched inulin, oligosaccharides, pectin, phosphate salts, phosphorus, polydextroses, polyols, potash, potassium, sodium nitrate, starch, sucrose, sulfur, sun fiber, tagatose, thiamine, trans-galactooligosaccharides, trehalose, vitamins, a water-soluble carbohydrate, and/or xylooligosaccharides (XOSs).

In some embodiments, the subject is characterized as having mucosal dysbiosis with LOH if the measured level of IL17C is characterized as elevated within the established IL17C level range, and one or more of IL17A, IL6, CXCL9, CCL11, CXCL11, and FGF23 is characterized as elevated within the established range of levels for each specific marker. In some embodiments, the subject is treated through the administration of a therapeutically effective amount of one or more antibiotic agents.

In some embodiments, the subject is characterized as having mucosal dysbiosis in the context of overt inflammation if the measured level of IL17C is characterized as elevated within the established IL17C level range, and one or more of CRP, SAA1 and S100A8 are characterized as elevated within the established range of levels for each specific marker. In some embodiments, the subject is treated through the administration of a therapeutically effective amount of one or more antibiotic agents in conjunction with anti-inflammatory and/or immunosuppressive therapy.

In some embodiments, the antibiotic is selected from the group consisting of: rifabutin, clarithromycin, clofazimine, vancomycin, rifampicin, nitroimidazole, chloramphenicol, and a combination thereof. In another aspect, an antibiotic composition administered herein comprises an antibiotic selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, and a combination thereof.

In certain embodiments, the present invention provides a kit comprising one or more of a prebiotic agent, a probiotic agent, a postbiotic agent, an antibiotic, and reagents capable of measuring one or more of IL17C, IL17A, IL6, CCL20, CXCL9, CCL11, CXCL11, FGF23, CRP, SAA1, and S100A8 levels within a biological sample.

### BRIEF DESCRIPTION OF DRAWINGS

FIG. 1A-B: Cell-autonomous induction of epithelial IL17c expression by exposure to gram-negative bacteria. (A) Differential microbiota-dependent regulation of IL17c, IL17a, and Reg3g (IL22 target gene) in the mouse intestine. GF, germ-free; CONV, conventionalized (SPF); SFB^{mono}, monocolonized with segmented filamentous bacteria. *, p<0.05; **, p<0.01 (Kruskal-Wallis with Dunn's post hoc test). (B) Acute cell-autonomous induction of IL17c expression in enteroid-derived epithelial monolayers directly exposed to bacteria. *, p<0.05; **, p<0.01 (Kruskal-Wallis with Dunn's post hoc test).

FIG. 2A-L: Mice with a defect in gut epithelial host defense are prone to IL17c induction in the intestinal mucosa linked to the expansion of gram-negative pathobionts. (A-B) IL17c mRNA expression in the terminal ileum and colon of Duoxa^{-/-} mice and wildtype (wt) littermates. Arrows indicate samples with outlier high IL17c expression (IL17c^{high}). Ccl20 (C), IL17a (D), and IL17f (E) expression in the terminal ileum. **, p<0.01; ***, p<0.001 (2-tailed Mann-Whitney). (F) To test whether IL17c induction in DUOX2 defective mice is dependent on the gut microbiota, mice were treated for three days with an antibiotics (Abx) regimen comprising ciprofloxacin and metronidazole (50 mg/kg b.w., bid by oral gavage). ***, p<0.001 (Kruskal-Wallis test), **, p<0.01 (Dunn's post hoc test). (G) To confirm the effect on the level of live, mucosa-associated microbiota, bacterial 16S rRNA level was determined in mucosal samples from the terminal ileum by amplification with universal eubacterial primers. Bacterial rRNA levels are normalized to the level of the mouse Hprt1 housekeeping gene. (H) Amplification with primers specific for □amma- and □elta-Proteobacteria. (I) Cladogram (phylum to genus level) depicting results of LEfSe (1) analysis identifying taxa with distinct relative abundance in ileal mucosa of Duoxa^{-/-} compared to wt littermates. (J) Discriminative taxa in the ileal mucosal microbiota of IL17c^{high} animals (arrows in A). (K) Relative mucosal abundance of Helicobacter (operational taxonomic unit [otu]0031). (L) Relative abundance of Proteobacterium otu0194 vs mucosal IL17c expression.

FIG. 3A-C: Proteobacterial otu0194 is detected in the mucosal niche of IL17C^{high} samples. (A) Ileal IL17c expression in wt and Duoxa^{-/-} mice derived from 14 distinct breeding pairs (parental genotypes: Duox^{+/-}). For each litter, mice were separated by genotype at weaning (P21). Five Duoxa^{-/-} mice had outlier high IL17c expression. (B) Relative abundance of otu0194 in ileal mucosal samples. (C) Relative abundance of otu0194 in corresponding luminal content of ileal samples.

FIG. 4A-C: T-cell independent induction of IL17c in the ileum of epithelial-specific Duoxa knockout mice. (A) Duoxa2 mRNA expression in the terminal ileum of intestinal epithelial-specific Duoxa knockout and floxed littermate control mice. **, p<0.01 (2-tailed Mann-Whitney). (B) Expression of IL17c in the ileum of intestinal epithelial-specific Duoxa knockout and floxed littermate control mice. (C), IL17c expression in Rag1^{-/-} mice lacking T cells as a major source of IL17 family cytokines. **, p<0.01; ***, p<0.001 (2-tailed Mann-Whitney).

FIG. 5: IL17c expression in the gut mucosa is highly responsive to impaired function of the supraepithelial mucus layer. The gut microbiota is separated from the mucosa by a supra-epithelial mucus layer that retains secreted antimicrobial effectors (antimicrobial peptides, secreted immunoglobulin A, H₂O₂). We challenged the normal bacterial compartmentalization by chronically feeding the emulsifier carboxymethylcellulose (CMC) that thins the mucus layer (2). *, p<0.05; **, p<0.01; ***, p<0.001 (Kruskal-Wallis and Dunn's post hoc test).

FIG. 6A-E: IL17C induction observed in a subset of IBD patients is a marker for abnormal epithelial stimulation by gram-negative bacteria. (A) Positive associations of plasma IL17C concentration with self-reported health history of 2,762 participants of a wellness program (Arivale) considering GI, lung, skin, and chronic infectious disease categories. Shown is the average difference in standardized plasma IL17C for presence vs absence of a condition. We evaluated the nominal significance of effects using Welch 2-sample test adjusted for age, sex, body mass index, season, and ancestry. (B) Expression of IL17C in ileal mucosal biopsies from patients with Crohn's disease (CD; n=174) and non-IBD controls (n=42) from the RISK cohort. (C) Gene set enrichment analysis using correlation with IL17C expression (r_{IL17C}) as rank metric to identify IL17C-correlated KEGG pathways in the mucosal biopsies of CD patients (FDR<0.05). (D) Overrepresentation of IL17C-coexpression signature (r_{IL17C}>0.5) in disease-associated gene sets from the GLAD4U database (3) (FDR<0.05). (E) Multivariate association analysis using the expression of IL17C and proinflammatory cytokines (TNF, IL1B) in ileal CD biopsies (n=135) as predictors and genus-level microbial abundance data of the mucosal microbiome as a response. Positive coefficients indicate a positive correlation between gene expression and compositional abundance of a bacterial genus.

FIG. 7A-F: Deleterious DUOX2 protein variants are associated with outlier high plasma IL17C concentration in the general population. (A) Frequency of rare (allele frequency [AF]<0.01) DUOX2/DUOXA2 protein variants identified among 2,762 participants of a wellness program (Arivale). Variants were classified using Variant Effect Predictor (VEP; Ensembl). (B) Most significant phenome-wide association results for rare DUOX2/DUOXA2 variants. p(SKAT-0) indicates the probability value of the SKAT-O test within each data category (proteins, metabolites, clinical labs, microbiome) selecting the optimal mixture of burden and variance component. FDR(SKAT-O) indicates the False Discovery Rate (FDR) corrected significance threshold across all data types. Full results of PheWAS are shown in Table 1 (C) Relative plasma IL17C baseline levels in study participants with or without DUOX2/DUOXA2 protein variants. NPX, Normalized Protein Expression (Olink assay). *, p<0.05 (2-tailed Kolmogorov-Smirnov test). (D) Prevalence of high IL17C level in subjects with or without DUOX2/DUOXA2 protein variants. We set the cut-off for outlier high IL17C level (IL17C^{high}) to Q3+2IQR of the no-variant group and stratified variants by rarity according to ancestry-specific allele frequency data from gnomAD. *, p<0.05 (2-tailed Fisher's exact test). (E) Identification of variants significantly contributing to the association with plasma concentration of IL17C in the study cohort (Wald chi-squared test). (F) H₂O₂-generating activity and targeting to the cell surface of DUOX2 variants expressed in a heterologous system (4). Data represent means±SEM; variants with significant loss-of-function are indicated by red color.

FIG. 8A-D: DUOX2 variants associated with IL17C^{high} confer increased risk for IBD. (A) Outline of the case-control study comparing the burden of high impact DUOX2 protein variants in IBD patients and ancestry-matched non-IBD control cohorts. We stratified variants using population-specific allele frequencies from the gnomAD database. (B) Contribution of individual high impact DUOX2 protein variants to the cumulative allele frequencies. NFE, Non-Finnish European; ASJ, Ashkenazi Jewish; FIN, Finnish. Note that the low prevalence of very rare variant carriers in Finns is due to multiple genetic bottlenecks in that isolated population (5). (C) Carriers of high impact DUOX2 protein variants are at increased risk for developing IBD. The Forest plot depicts estimated odds ratios (OR) with 95% CI for UC and CD patients from the three ancestry cohorts. The combined OR was calculated using a random-effects model with the Mantel-Haenszel weighting method. **, p<0.01; ***, p<0.001 (test of null hypothesis that odds ratio is equal to 1) (6). (D) Detailed view of DUOX2 variants with predicted complete loss-of-function (i.e. frameshift, stop gained, and splice donor or acceptor site variants) in IBD and control cohorts. *, p<0.05 (2-tailed Fisher's exact test).

FIG. 9A-C: Identification of candidate microinflammation markers in subjects with mucosal dysbiosis (IL17C^{high} subjects). (A) The plasma level of 91 inflammation-related proteins was compared between subjects with outlier high plasma IL17C level (IL17C^{high}: 99^{th} percentile for IL17C; n=27) and those with normal/low plasma IL17C (<95^{th} percentile for IL17C; n=2580). The vulcan plot shows the geometric mean ratio (GMR; 99^{th} vs<95^{th} percentile for IL17C) on the x-axis and the significance level (FDR; 2-tailed Mann-Whitney test with Benjamini-Hochberg correction) on the y-axis. The GMR(log ₂) for IL17C was 2.28 (not shown). Note that only one of the IL17C^{high} subjects had (self-reported) IBD; exclusion of data for this subject did not meaningfully change the overall protein profile. (B) Analysis of the plasma protein profile in CD patients and non-IBD controls. Protein level data were obtained from a study by Andersson et al. (7). The vulcan plot depicts the geometric mean ratios (CD vs non-IBD controls; log ₂) on the x-axis and the corresponding FDR values on the y-axis. (C) IL17C^{high} status is associated with specific alterations of the plasma protein profile that are not unique to carriers of DUOX2 protein variants. The plasma level of 91 inflammation-related proteins was analyzed in subjects with outlier high plasma IL17C level (IL17C^{high}: 99^{th} percentile for IL17C; n=27). Plotted are the relative protein levels in IL17C^{high} subjects with (y-axis; n=13) or without (x-axis; n=14) rare DUOX2 protein variant. Protein levels are expressed as geometric mean ratio (GMR) relative to the total study cohort.

FIG. 10: Exemplar implementation of IL17C as a dysbiosis marker in a multiplex biomarker assay to guide diagnostic and therapeutic decisions in at-risk individuals.

### DETAILED DESCRIPTION OF THE INVENTION

The present invention provides methods for detecting intestinal inflammation (e.g., intestinal microinflammation, intestinal microinflammation) and treating and/or preventing elevated intestinal inflammation through, for example, measuring a level of interleukin 17C (IL17C) in a biological sample from a subject, and if elevated, treating and/or preventing such intestinal inflammation.

In certain embodiments, the present invention provides methods comprising:
measuring an IL17C level in a biological sample obtained from a subject;
characterizing the measured IL17C level within an established IL17C range;
measuring the levels of interleukin 17A (IL17A), interleukin 6 (IL6), C—C motif chemokine ligand 20 (CCL20), C—X—C motif chemokine ligand 9 (CXCL9), C—C Motif Chemokine Ligand 11 (CCL11), C—X—C motif chemokine ligand 11 (CXCL11), Fibroblast growth factor-23 (FGF23), C-reactive protein (CRP), serum amyloid A (SAA1), and neutrophilic marker calprotectin (S100A8) within the biological sample if the measured IL17C level is characterized as elevated within the established IL17C range;
characterizing the measured IL17A, IL6, CCL20, CXCL9, CCL11, CXCL11, FGF23, CRP, and S100A8 levels within established ranges for IL17C, IL17A, IL6, CCL20, CXCL9, CCL11, CXCL11, FGF23, CRP, SAA1, and S100A8 levels;
characterizing an intestinal inflammation status for the subject based upon the characterized IL17C, IL17A, IL6, CCL20, CXCL9, CCL11, CXCL11, FGF23, CRP, SAA1, and S100A8 levels; and
treating the characterized intestinal inflammation status in the subject.

Such methods are not limited to a particular type of subject. As used herein, “subject” refers to any animal subject including humans, laboratory animals (e.g., primates, rats, mice), livestock (e.g., cows, sheep, goats, pigs, turkeys, chickens), and household pets (e.g., dogs, cats, rodents, etc.).

In some embodiments, the subject is a mammal. In some embodiments, the subject is a human. In some embodiments, the subject is a human subject suffering or at risk of suffering from a breakdown of microbiota/immune system homeostasis. In some embodiments, the subject is a human subject suffering or at risk of suffering from an expansion of proteobacteria pathobionts. In some embodiments, the subject is a human subject suffering or at risk of suffering from inflammatory bowel disease (IBD) due to a loss of microbiota/immune system homeostasis at gut epithelial surfaces. In some embodiments, the subject is a human subject who has IBD, is diagnosed with IBD, is suspected to have IBD, is likely to have IBD, has one or more signs or symptoms of IBD (e.g., gastrointestinal, systemic, and extraintestinal symptoms), has increased risk for developing IBD based on positive family history or the presence of one or more risk variants in IBD susceptibility genes. In some embodiments, the subject is a human subject who has been previously diagnosed with irritable bowel syndrome (IBS), obesity, metabolic syndrome, hepatic encephalopathy, colon cancer.

Such methods are not limited to a particular biological sample. In some embodiments, the biological sample is a blood sample (e.g., plasma, serum, whole blood). In some embodiments, the biological sample is a tissue sample (e.g., an intestinal tissue sample).

Such methods are not limited to a particular manner of measuring IL17C, IL17A, IL6, CCL20, CXCL9, CCL11, CXCL11, FGF23, CRP, SAA1, and S100A8 levels in the biological sample.

Such methods are not limited to a particular manner of characterizing the measured IL17C, IL17A, IL6, CCL20, CXCL9, CCL11, CXCL11, FGF23, CRP, SAA1, and S100A8 levels within established ranges for respective marker (IL17C, IL17A, IL6, CCL20, CXCL9, CCL11, CXCL11, FGF23, CRP, SAA1, and S100A8) levels. In some embodiments, the established marker range is an established range of levels for that specific marker generated from a plurality of subjects (e.g., human subjects) (e.g., human subjects not suffering from intestinal inflammation and human subjects suffering from intestinal inflammation). In some embodiments, the measured marker level is compared with the established range of levels for that specific marker such that a percentage of the established range of levels for that specific marker is obtained (e.g., bottom 1% of the specific marker levels, bottom 5% of the specific marker levels, bottom 10%, 20%, 30%, 40%, etc.) (e.g., top 1% of the specific marker levels, top 5% of the specific marker levels, top 10%, 20%, 30%, 40%, etc.).

Such methods are not limited to particular manner of establishing if a measured marker is characterized as elevated within the established range of that marker. In some embodiments, a characterization of the measured marker within the top 45% of established range of that marker is elevated. In some embodiments, a characterization of the measured marker within the top 40% of established range of that marker is elevated. In some embodiments, a characterization of the measured marker within the top 30% of established range of that marker is elevated. In some embodiments, a characterization of the measured marker within the top 35% of established range of that marker is elevated. In some embodiments, a characterization of the measured marker within the top 25% of established range of that marker is elevated. In some embodiments, a characterization of the measured marker within the top 20% of established range of that marker is elevated. In some embodiments, a characterization of the measured marker within the top 15% of established range of that marker is elevated. In some embodiments, a characterization of the measured marker within the top 10% of established range of that marker is elevated. In some embodiments, a characterization of the measured marker within the top 8% of established range of that marker is elevated. In some embodiments, a characterization of the measured marker within the top 7% of established range of that marker is elevated. In some embodiments, a characterization of the measured marker within the top 6% of established range of that marker is elevated. In some embodiments, a characterization of the measured marker within the top 5% of established range of that marker is elevated. In some embodiments, a characterization of the measured marker within the top 4% of established range of that marker is elevated. In some embodiments, a characterization of the measured marker within the top 3% of established range of that marker is elevated. In some embodiments, a characterization of the measured marker within the top 2% of established range of that marker is elevated. In some embodiments, a characterization of the measured marker within the top 1% of established range of that marker is elevated.

Such methods are not limited to a particular manner of characterizing an intestinal inflammation status for the subject based upon the characterized marker levels.

In some embodiments, the subject is characterized as not having intestinal mucosal dysbiosis if the measured levels of IL17C are characterized as not elevated in comparison with the established IL17C level.

In some embodiments, the subject is characterized as having mucosal dysbiosis without loss of homeostasis if the measured level of IL17C is characterized as elevated within the established IL17C level range, and each of IL17A, IL6, CXCL9, CCL11, CXCL11, FGF23, CRP, SAA1, and S100A8 is characterized as not elevated within the established range of levels for each specific marker.

In some embodiments, the subject is characterized as having proinflammatory mucosal dysbiosis with loss of homeostasis (microinflammation) if the measured level of IL17C is characterized as elevated within the established IL17C level range, and one or more of IL17A, IL6, CXCL9, CCL11, CXCL11, and FGF23 is characterized as elevated within the established range of levels for each specific marker.

In some embodiments, the subject is characterized as having proinflammatory mucosal dysbiosis with overt inflammation if the measured level of IL17C is characterized as elevated within the established IL17C level range, and one or more of CRP, SAA1, and S100A8 is characterized as elevated within the established range of levels for each specific marker.

Such methods are not limited to a particular manner of treating a subject characterized as having elevated IL17C but not having intestinal inflammation. As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures.

In some embodiments, a subject characterized as having elevated IL17C but not having intestinal inflammation is treated through administration of a therapeutically effective amount of one or more agents selected from a prebiotic agent, a probiotic agent, and a postbiotic agent. In some embodiments, the agent is capable of restoring a state of intestinal in the subject.

In some embodiments, the prebiotic agent is selected from the group consisting of: complex carbohydrates, complex sugars, resistant dextrins, resistant starch, amino acids, peptides, nutritional compounds, biotin, polydextrose, fructooligosaccharide (FOS), galactooligosaccharides (GOS), inulin, starch, lignin, psyllium, chitin, chitosan, gums (e.g. guar gum), high amylose cornstarch (HAS), cellulose, s-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, xylooligosaccharides (XOS), locust bean gum, β-glucans, methylcellulose, and any combination thereof. In some embodiments, the prebiotic agent is an oligosaccharide. In some embodiments, the prebiotic agent is inulin.

In some embodiments, the prebiotic agent is selected from the group consisting of: amino acids, ammonium nitrate, amylose, barley mulch, biotin, carbonate, cellulose, chitin, choline, fructooligosaccharides (FOSs), fructose, galactooligosaccharides (GOSs), glucose, glycerol, heteropolysaccharide, histidine, homopolysaccharide, hydroxyapatite, inulin, isomaltulose, lactose, lactulose, maltodextrins, maltose, mannooligosaccharides, tagatose, nitrogen, oligodextrose, oligofructoses, oligofructose-enriched inulin, oligosaccharides, pectin, phosphate salts, phosphorus, polydextroses, polyols, potash, potassium, sodium nitrate, starch, sucrose, sulfur, sun fiber, tagatose, thiamine, trans-galactooligosaccharides, trehalose, vitamins, a water-soluble carbohydrate, and/or xylooligosaccharides (XOSs).

In some embodiments, the prebiotic agent, probiotic agent, and/or postbiotic agent is administered for at least 1 hour, 2 hours, 5 hours, 12 hours, 1 day, 3 days, 1 week, 2 weeks, 1 month, 6 months, or 1 year.

As used herein, “therapeutically effective amount” or “pharmaceutically active dose” refers to an amount of a composition which is effective in treating the named disease, disorder, or condition.

Such methods are not limited to a particular manner of treating a subject characterized as having intestinal inflammation. In some embodiments, a subject characterized as having intestinal inflammation is treated through administration of a therapeutically effective amount of one or more antibiotic agents. In some embodiments, the antibiotic agent is capable of restoring a state of intestinal eubiosis in the subject. As used herein, “antibiotic” refers to a substance that is used to treat and/or prevent bacterial infection by killing bacteria, inhibiting the growth of bacteria, or reducing the viability of bacteria.

In some embodiments, the antibiotic is selected from the group consisting of rifabutin, clarithromycin, clofazimine, vancomycin, rifampicin, nitroimidazole, chloramphenicol, and a combination thereof. In another aspect, an antibiotic composition administered herein comprises an antibiotic selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, and a combination thereof.

In some embodiments, the antibiotic agent is administered for at least 1 hour, 2 hours, 5 hours, 12 hours, 1 day, 3 days, 1 week, 2 weeks, 1 month, 6 months, or 1 year.

In some embodiments, an elevated IL17C level with or without an elevated CCL20 level results from or more mutations in the DUOX2 gene and/or the DUOX2 gene product. In some embodiments, one or more mutations in the DUOX2 gene encodes a loss of function mutation, deletion mutation, insertion mutation, splice acceptor mutation, splice donor mutation, and/or a gain of function mutation.

In some embodiments, the administering comprises administration of a pharmaceutical composition (e.g., comprising prebiotic agent, a probiotic agent, and a postbiotic agent, and/or antibiotic), orally, by enema, by injection, or via rectal suppository. In one aspect, a pharmaceutical composition administered herein is formulated as an enteric coated (and/or acid-resistant) capsule or microcapsule, or formulated as part of or administered together with a food, a food additive, a dairy-based product, a soy-based product, or a derivative thereof, a jelly, flavored liquid, ice block, ice cream, or a yogurt. In another aspect, a pharmaceutical composition administered herein is formulated as an acid-resistant enteric-coated capsule. A pharmaceutical composition can be provided as a powder for sale in combination with a food or drink. A food or drink can be a dairy-based product or a soy-based product. In another aspect, a food or food supplement contains enteric-coated and/or acid-resistant microcapsules containing a pharmaceutical composition.

In some embodiments, the pharmaceutical composition comprises a liquid culture. In another aspect, a pharmaceutical composition (e.g., comprising prebiotic agent, a probiotic agent, and a postbiotic agent, and/or antibiotic) is homogenized, lyophilized, pulverized, and powdered. It may then be infused, dissolved such as in saline, as an enema. Alternatively, the powder may be encapsulated as enteric-coated and/or acid-resistant delayed-release capsules for oral administration. In an aspect, the powder may be double encapsulated with acid-resistant/delayed-release capsules for oral administration. These capsules may take the form of enteric-coated and/or acid-resistant delayed-release microcapsules. A powder can preferably be provided in a palatable form for reconstitution for drinking or for reconstitution as a food additive. In a further aspect, a food is a yogurt. In one aspect, a powder may be reconstituted to be infused via naso-duodenal infusion.

In some embodiments, the pharmaceutical composition (e.g., comprising prebiotic agent, a probiotic agent, and a postbiotic agent, and/or antibiotic) is administered herein is in a liquid, frozen, freeze-dried, spray-dried, foam-dried, lyophilized, or powder form. In a further aspect, a pharmaceutical composition administered herein is formulated as a delayed or gradual enteric release form. In another aspect, a pharmaceutical composition administered herein comprises an excipient, a saline, a buffer, a buffering agent, or a fluid-glucose-cellobiose agar (RGCA) media. In another aspect, a pharmaceutical composition administered herein comprises a cryoprotectant. In one aspect, a cryoprotectant comprises polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof.

In some embodiments, the pharmaceutical composition can be provided together with a pharmaceutically acceptable carrier. As used herein, a “pharmaceutically acceptable carrier” refers to a non-toxic solvent, dispersant, excipient, adjuvant, or other material which is mixed with a live bacterium in order to permit the formation of a pharmaceutical composition, e.g., a dosage form capable of administration to the patient. A pharmaceutically acceptable carrier can be liquid (e.g., saline), gel or solid form of diluents, adjuvant, excipients, or an acid-resistant encapsulated ingredient. Suitable diluents and excipients include pharmaceutical grades of physiological saline, dextrose, glycerol, mannitol, lactose, starch, magnesium stearate, sodium saccharin, cellulose, magnesium carbonate, and the like, and combinations thereof. In another aspect, a pharmaceutical composition may contain auxiliary substances such as wetting or emulsifying agents, stabilizing or pH buffering agents. In an aspect, a pharmaceutical composition contains about 1%-5%, 5%-10%, 10%-15%, 15-20%, 20%-25%, 25-30%, 30-35%, 40-45%, 50%-55%, 1%-95%, 2%-95%, 5%-95%, 10%-95%, 15%-95%, 20%-95%, 25%-95%, 30%-95%, 35%-95%, 40%-95%, 45%-95%, 50%-95%, 55%-95%, 60%-95%, 65%-95%, 70%-95%, 45%-95%, 80%-95%, or 85%-95% of active ingredient. In an aspect, a pharmaceutical composition contains about 2%-70%, 5%-60%, 10%-50%, 15%-40%, 20%-30%, 25%-60%, 30%-60%, or 35%-60% of active ingredient.

In some embodiments, the pharmaceutical composition can be incorporated into tablets, drenches, boluses, capsules, or premixes. Formulation of these active ingredients into such dosage forms can be accomplished by means of methods well known in the pharmaceutical formulation arts. See, e.g., U.S. Pat. No. 4,394,377. Filling gelatin capsules with any desired form of the active ingredients readily produces capsules. If desired, these materials can be diluted with an inert powdered diluent, such as sugar, starch, powdered milk, purified crystalline cellulose, or the like to increase the volume for convenience of filling capsules.

In some embodiments, conventional formulation processes can be used to prepare tablets containing a pharmaceutical composition. In addition to the active ingredients, tablets may contain a base, a disintegrator, an absorbent, a binder, and a lubricant. Typical bases include lactose, sugar, sodium chloride, starch, and mannitol. Starch is also a good disintegrator as is alginic acid. Surface-active agents such as sodium lauryl sulfate and dioctyl sodium sulphosuccinate are also sometimes used. Commonly used absorbents include starch and lactose. Magnesium carbonate is also useful for oily substances. As a binder there can be used, for example, gelatin, gums, starch, dextrin, polyvinyl pyrrolidone, and various cellulose derivatives. Among the commonly used lubricants are magnesium stearate, talc, paraffin wax, various metallic soaps, and polyethylene glycol.

In some embodiments, for preparing solid compositions such as tablets, an active ingredient is mixed with a pharmaceutical carrier, e.g., conventional tableting ingredients such as corn starch, lactose, sucrose, sorbitol, talc, stearic acid, magnesium stearate, dicalcium phosphate, or gums, or other pharmaceutical diluents, e.g. water, to form a solid preformulation composition containing a homogeneous mixture of a composition of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing a desired amount of an active ingredient (e.g., at least about 105, 10⁶, 107, 10¹, 10¹, 10¹⁰, 10¹¹, 10¹², or 10¹³ cfu). A pharmaceutical composition used herein can be flavored.

In some embodiments, a pharmaceutical composition can be a tablet or a pill. In one aspect, a tablet or a pill can be coated or otherwise compounded to provide a dosage form affording the advantage of prolonged action. For example, a tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

In some embodiments, a pharmaceutical composition can be a drench. In one aspect, a drench is prepared by choosing a saline-suspended form of a pharmaceutical composition. A water-soluble form of one ingredient can be used in conjunction with a water-insoluble form of the other by preparing a suspension of one with an aqueous solution of the other. Water-insoluble forms of either active ingredient may be prepared as a suspension or in some physiologically acceptable solvent such as polyethylene glycol. Suspensions of water-insoluble forms of either active ingredient can be prepared in oils such as peanut, corn, sesame oil or the like; in a glycol such as propylene glycol or a polyethylene glycol; or in water depending on the solubility of a particular active ingredient. Suitable physiologically acceptable adjuvants may be necessary in order to keep the active ingredients suspended. Adjuvants can include and be chosen from among the thickeners, such as carboxymethylcellulose, polyvinyl pyrrolidone, gelatin and the alginates. Surfactants generally will serve to suspend the active ingredients, particularly the fat-soluble propionate-enhancing compounds. Most useful for making suspensions in liquid nonsolvents are alkylphenol polyethylene oxide adducts, naphthalenesulfonates, alkylbenzene-sulfonates, and the polyoxyethylene sorbitan esters. In addition, many substances, which affect the hydrophilicity, density, and surface tension of the liquid, can assist in making suspensions in individual cases. For example, silicone anti-foams, glycols, sorbitol, and sugars can be useful suspending agents.

In certain embodiments, the present invention provides kits comprising one or more of a prebiotic agent, a probiotic agent, a postbiotic agent, an antibiotic, and reagents capable of measuring one or more of IL17C, IL17A, IL6, CCL20, CXCL9, CCL11, CXCL11, FGF23, CRP, and S100A8 levels within a biological sample.

A featured kit comprises reagents capable of measuring levels within a biological sample of (1) IL17C; (2) one or more biomarkers of loss of gut epithelial homeostasis such as IL17A, IL6, CXCL9, CCL11, CXCL11, and FGF23; and (3) one or more biomarkers of overt gut epithelial inflammation such as CRP, SAA1, and S100A8. The kit may further include: (4) treatment for (i) micro-inflammatory gut dysbiosis, (ii) macro-inflammatory gut dysbiosis, or (iii) combinations of thereof.

As such, in some embodiments a method of treating gut dysbiosis in a subject is provided, comprising: (a) measuring the levels of first and second proteins in a blood and/or tissue sample of the subject, the first protein interleukin 17C (IL17C) and the second protein depicting the intestinal inflammation status of the subject selected from (i) a biomarker of loss of gut epithelial homeostasis, (ii) a biomarker of overt gut epithelial inflammation, and (iii) combinations of (i) and (ii); and (b) treating the subject for (i) micro-inflammatory gut dysbiosis when the subject is characterized as having elevated levels of the first and second proteins relative to an established range, the second protein a biomarker of a loss of gut epithelial homeostasis, or (ii) macro-inflammatory gut dysbiosis when the subject is characterized as having elevated levels of the first and second proteins relative to an established range, the second protein a biomarker of overt gut epithelial inflammation.

Of specific interest is where the first protein further includes C—C motif chemokine ligand 20 (CCL20); the biomarker of loss of gut epithelial homeostasis is selected from IL17A, IL6, CXCL9, CCL11, CXCL11, and FGF23; and the biomarker of overt gut epithelial inflammation is selected from CRP, SAA1, and S100A8. In as many embodiments, the (a) gut epithelial homeostasis is characterized by normal levels of inflammation biomarker proteins IL17A, IL6, CXCL9, CCL11, CXCL11, FGF23, and (b) gut epithelial inflammation is characterized by normal levels of CRP, SAA1, and S100A8. In certain embodiments, the treatment for micro-inflammatory gut dysbiosis is selected from prebiotics, probiotics, and antibiotics; and the treatment for macro-inflammatory gut dysbiosis is standard IBD treatment.

One of ordinary skill in the art will readily recognize that the foregoing represents merely a detailed description of certain preferred embodiments of the present invention. Various modifications and alterations of the compositions and methods described above can readily be achieved using the expertise available in the art and are within the scope of the invention.

### Experimental

The following examples are illustrative, but not limiting, of the compositions, and methods of the present invention. Other suitable modifications and adaptations of the variety of conditions and parameters normally encountered in clinical therapy and which are obvious to those skilled in the art are within the spirit and scope of the invention. The terms “we,” “I”, and “our” refer to the inventors for this technology.

### Example I

This example describes the identification of IL17C as a biomarker for disturbed gut microbe-epithelial interaction.

### Methods

### Acute Microbial Exposure of Colonoid-Derived Monolayers.

Colonoids and colonoid-derived monolayers from Duoxa^{-/-} mice (n=3) and wild-type littermates (n=3) were established following previously outlined protocols (8). For acute exposure to bacteria, the culture medium was replaced by HBSS(Ca²⁺) supplemented with 20 mM HEPES, 10 mM glucose, and 1% FBS. Bacteria (Salmonella Typhimurium strain SL1344, Citrobacter rodentium strain DBS120, Escherichia coli strain K12, Enterococcus faecalis (mouse cecum-derived isolate), Lactobacillus rhamnosus GG, Clostridium scindens (9)) were washed in the same buffer and added at MOI ˜10 to the apical compartment. For experiments under anaerobic conditions, cell monolayers and buffer were pre-equilibrated for 1 h.

### Real-Time Reverse Transcription PCR (RT-qPCR).

Total RNA extractions were prepared using TRIzol reagent, treated with deoxyribonuclease, and cleaned up on RNeasy spin columns (Qiagen). RNA was reverse transcribed with Superscript II (Life Technologies) using random hexamer priming. qPCR was performed as previously described (10). Amplification specificity was confirmed by melting curve analysis of products and gene expression was normalized to Hprt1 mRNA.

### Animals.

Duoxa^{-/-} mice lacking functional DUOX enzymes have been described previously (11). Rag1^{-/-} (Rag1^{tm1Mom}) (6) in the C57BL/6 background were used to generate Duoxa^{flox/flox} mice deficient in T and B cells. All animal studies were approved by the University of Michigan Institutional Animal Care and Use Committee (PRO-00007922).

### Gut Microbiota Manipulations.

GF mice were orally gavaged with a freshly prepared suspension of frozen cecal material from mice monocolonized with SFB (12), SPF mice, or GF controls. CMC was dissolved at 1% (w/v) concentration in drinking water. Treatment was initiated at weaning and continued with weekly solution changes for 8 weeks (P21-P77). 16S rDNA profiles from mouse mucosal samples.

Genomic DNA was extracted using a modified protocol of the Qiagen DNeasy Blood & Tissue kit that included an initial bead-beating step (0.7 mm garnet) for cell wall disruption. 16S rRNA gene libraries were constructed using primers specific to the V4 region and subjected to Illumina MiSeq 250 bp paired-end sequencing. FASTQ files have been deposited in the NCBI Sequence Read Archive under BioProject PRJNA590250. Sequences were curated using the mothur v1.40.5 (13) pipeline implemented in Nephele (v2.2.8) (14). Sequences were assigned to operational taxonomic units (OTUs) using a dissimilarity cutoff=0.03 and classified against the nonredundant SILVA v128 ribosomal RNA database.

Correlation of Host Gene Expression Level with Microbial Abundance Data.

LEfSe (linear discriminant effect size) analysis (1) was used to identify taxa distinguishing IL17C^{high} and IL17C^{low} microbiota based on significance level and estimated effect size. Boosted additive general linear models between multiple host predictors and arcsin-square root transformed relative abundance data of the mucosal microbiome as a response were calculated using MaAsLin (15).

### Statistics.

As indicated, we evaluated group differences for statistical significance with one-way ANOVA with Dunnett's multiple comparisons test (>2 groups; parametric), Kruskal-Wallis test with Dunn's post-hoc test (>2 groups; non-parametric), Mann-Whitney (2 groups; non-parametric), or Fisher's exact test (contingency tables). Data were analyzed with GraphPad Prism 8.0 (San Diego, CA). We used Meta-Essentials (16) to assess genetic risk from allele count data and WebGestalt 2017 (17) for gene set enrichment and overrepresentation analyses.

### Results

### 1) Gut Epithelial IL17C Expression is Silenced in Healthy, Eubiotic Mice but can be Cell-Autonomously Induced by Direct Exposure to Gram-Negative Bacteria.

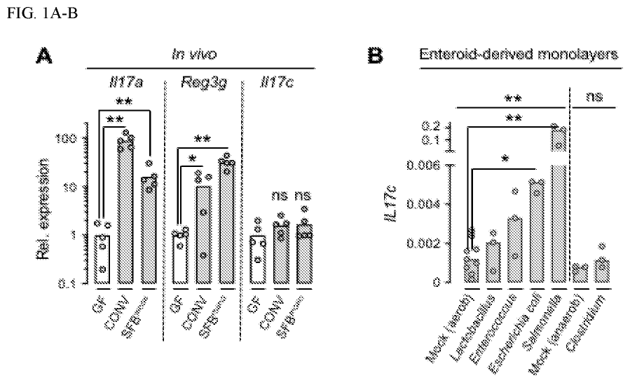

We found that in vivo, colonization of germ-free mice with microbiota from specific-pathogen-free (SPF) mice or monocolonization with Segmented Filamentous Bacteria (SFB; epithelial-attaching, gram-positive bacteria) failed to significantly induce IL17c (FIG. 1A). This was in contrast to the well-known activation of other epithelial defense systems (e.g., Duox2, Reg3g) and their cognate inducers, such as IL22 and IL17a under these conditions (18, 19). Thus, IL17c is not significantly activated by any of the signaling pathways upregulated in response to conventionalization of axenic animals (20). In contrast and consistent with cell-autonomous regulation by direct contact microbiota, we found that in epithelial cell monolayers derived from mouse colonoids, IL17c expression was rapidly induced by direct exposure to gram-negative Enterobacteriaceae, but not the gram-positive bacteria tested (FIG. 1B). The latter results were also in agreement with published data indicating that IL17c expression is acutely upregulated in epithelial cell lines by stimulation with the Toll-like receptor 5 ligand flagellin (21).

2) Mice with a Defect in Gut Epithelial Host Defense are Prone to IL17c Induction in the Intestinal Mucosa Linked to the Expansion of Gram-Negative Pathobionts.

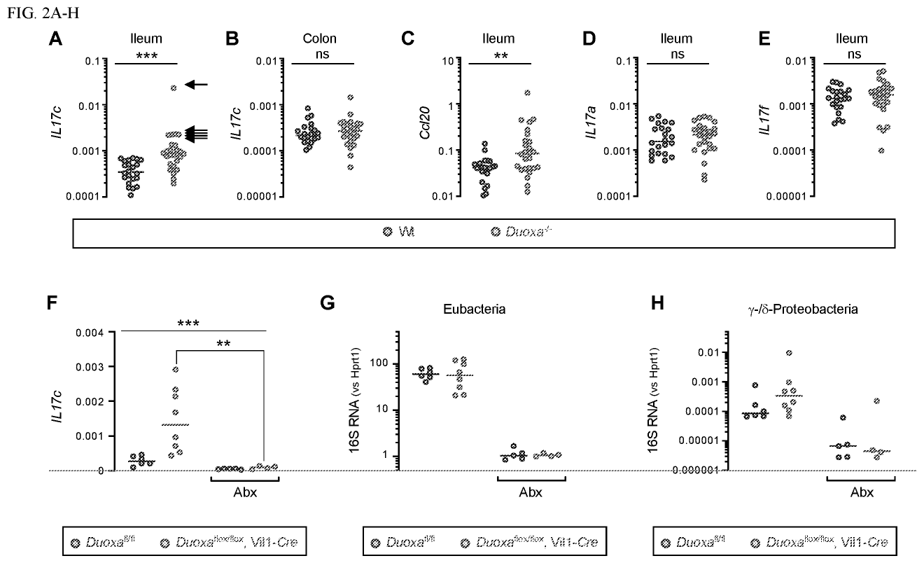

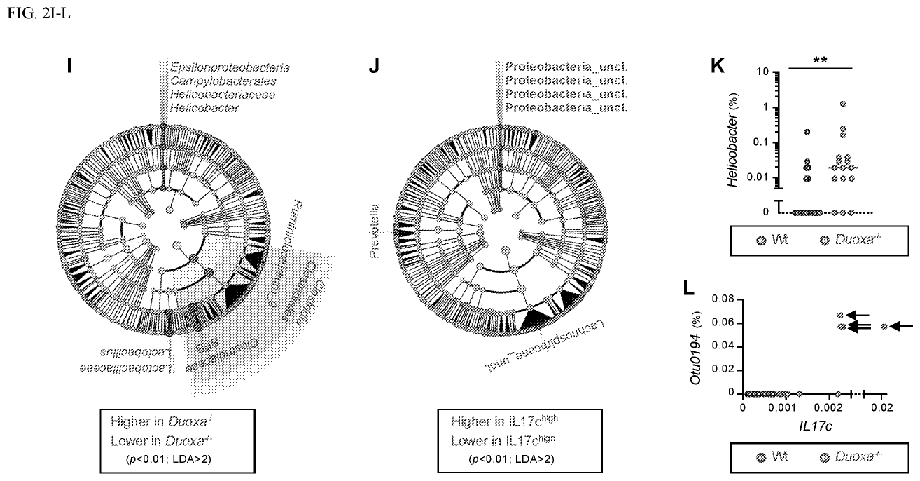

We next explored the regulation of IL17C in mice with genetic deletion of the hydrogen-peroxide generating epithelial NADPH oxidase (DUOX2/DUOXA2 heterodimeric enzyme), which provides an anti-microbial host-defense system at the apical surface of the gut epithelium. DUOX2 inactivation alone is not sufficient to trigger spontaneous gut inflammation. However, compared to wild-type littermates, both Duoxa^{-/-} mice lacking Duox2 activity had significantly higher IL17c expression in the mucosa of the terminal ileum, but not the colon (FIGS. 2A and 2B). Approximately 15% of knockout mice had outlier high IL17c expression in the ileum (IL17C^{high}; arrows in FIG. 2A). We found that IL17c induction was accompanied by significantly higher tissue expression of the chemokine Ccl20, but not of IL17a or IL17f (FIGS. 2C-2E), suggesting activation of an epithelial innate immune response, but not of the lymphocyte compartment.

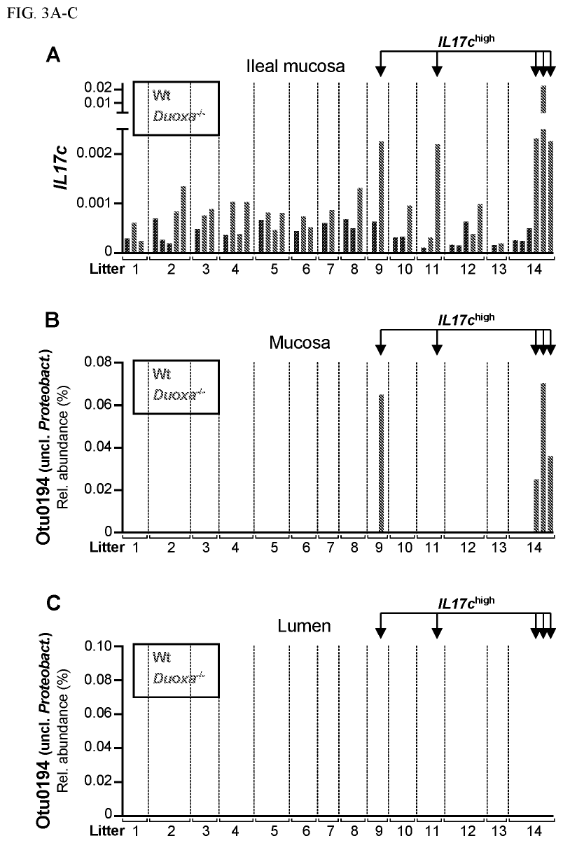

It is plausible that a defect in hydrogen peroxide release from the apical membrane of enterocytes increases access of susceptible gram-negative bacteria to the epithelium, for instance, due to reduced chemorepulsive, virulence-suppressing, or bactericidal effects (10, 22, 23). Furthermore, a stochastic shift in mucosal microbiota composition with an expansion of specific gram-negative pathobionts could underlie excessive IL17c levels found in a subset of Duoxa-deficient mice. Therefore, we profiled the composition of the ileal mucosal microbiota by 16S rDNA sequencing. Compared to wild-type littermates, Duoxa^{-/-} mice had altered mucosal microbiota composition characterized by a relative loss of SFB with correspondingly higher abundance of Helicobacter and Lactobacillus (FIGS. 2F and 2H). The most discriminative feature in IL17C^{high} mice (arrows in FIG. 2A) was an unclassified Proteobacterium (Otu0194) (FIGS. 2G and 2I; Table 4). Otu0194 was also the most significant IL17c-associated taxonomic feature after adjusting for Duoxa genotype (FDR=0.0065; Table 2). The mucosal niche appeared to be its preferred habitat since it was not detected by sequencing of the corresponding luminal samples (FIG. 3).

3) Induction of IL17c in the Gut of Mice with Epithelial Host Defense Defect is T-Cell Independent.

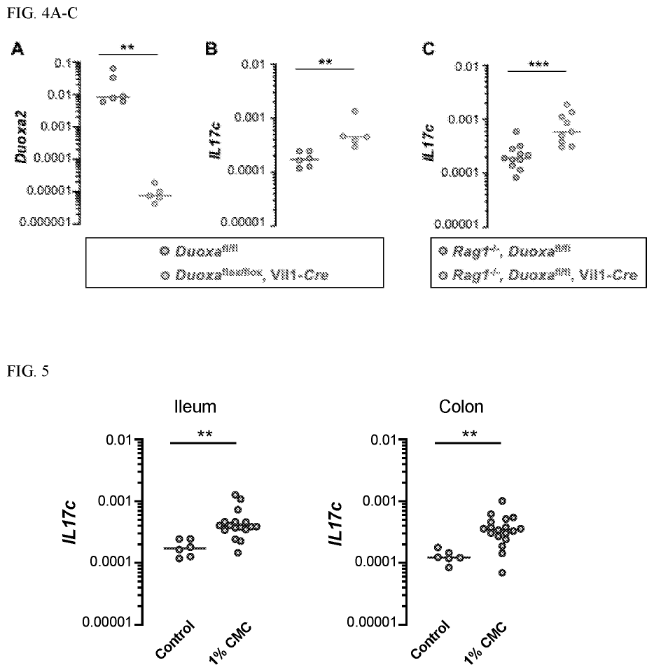

This phenotype of mice lacking intestinal Duox2 activity was also completely T-cell independent since it was conserved in a T (and B) cell-deficient Rag^{-/-} background (FIG. 4). The finding of abnormal IL17c expression in the ileum but not colon is consistent with the relatively higher baseline expression of Duox2 in the ileum of mice kept in a specific-pathogen-free (SPF) environment (18).

4) IL17c Expression in the Gut Mucosa is Highly Responsive to Impaired Function of the Supraepithelial Mucus Layer Separating the Microbiota from the Epithelium.

In addition to secreted compounds such as antimicrobial peptides and DUOX2-generated hydrogen peroxide, the supraepithelial mucus layer provides an important physical barrier preventing contact between the luminal microbiota and the epithelium in healthy conditions. In the colon, the thick inner mucus layer is essentially sterile, whereas the thinner non-stratified mucus layer of the ileum is more readily penetrable by bacteria-sized particles, but nevertheless important for the effectiveness of antimicrobial compounds by limiting their diffusion into the lumen (24). The thickness of the mucus layer can be affected by dietary factors. For instance, intake of emulsifiers such as carboxymethylcellulose (aka cellulose gum) that are widely used in the preparation of processed foods, have been shown to cause thinning of the protective mucus layer (2). We found that feeding mice a moderate concentration of 1% carboxymethylcellulose robustly induced IL17C without induction of other inflammatory markers indicating that its expression is a remarkably sensitive marker for excessive exposure of the epithelium to microbiota (FIG. 5).

### 5) In IBD Patients, IL17C Induction is a Marker for Abnormal Epithelial Stimulation by Gram-Negative Mucosal Dysbiosis.

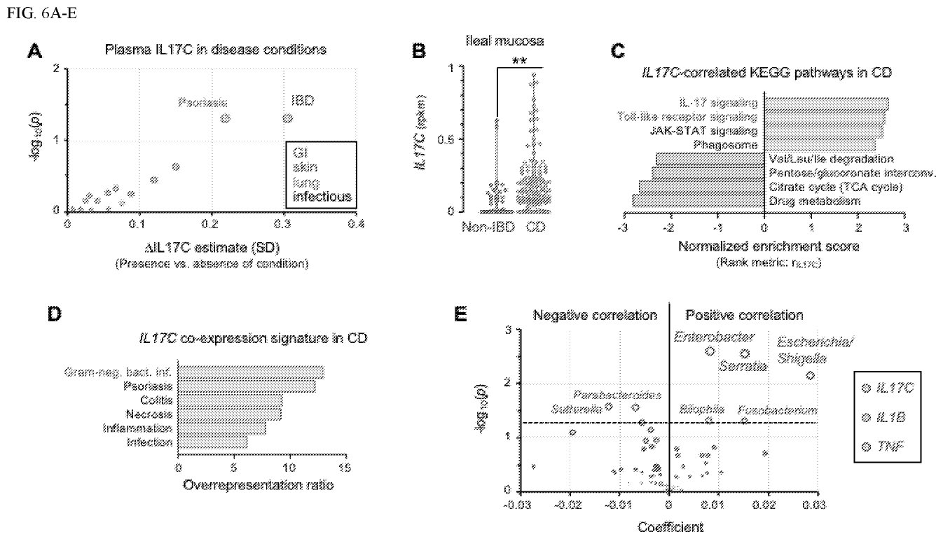

To examine the potential of IL17C as a biomarker for disturbed gut microbe-immune homeostasis in humans, we modeled the baseline plasma IL17C concentrations of 2,762 participants of a lifestyle coaching program (Arivale) on self-reported health history conditions. This analysis revealed that a diagnosis of IBD was strongest associated with elevated plasma IL17C level (FIG. 6A). Furthermore, we found that ileal IL17C expression in treatment naïve Crohn's Disease (CD) patients from the RISK Cohort Study (25, 26) was indeed more frequently induced compared to controls without IBD (FIG. 6B). Analysis of gene expression profiles revealed that the pathways most strongly associated with IL17C induction were linked to anti-bacterial response with the leading IL17C-correlated genes being the strongest implicated in gram-negative bacterial infections (FIGS. 6C and 6D).

To further explore the potential of IL17C as a specific and sensitive sentinel response to mucosal dysbiosis, we performed an integrated analysis of matched host transcriptome and 16S rRNA sequencing data (RISK cohort; Table 6). The mucosal microbiota in the ileum of these CD patients is primarily characterized by a higher relative abundance of Proteobacteria of the Enterobacteriaceae and Neisseriaceae families (26). Though these characteristic shifts in the ileal microbial composition are to some degree observed in colonic CD patients without overt ileal inflammation (25), there is also a well-established interdependency between the bloom of Enterobacteriaceae and the inflammatory environment (27). Thus, to test whether the induction ofIL17C is a predictor of epithelial activation by mucosal dysbiosis, we performed multivariate association analysis using IL17C and IBD-associated proinflammatory cytokines (TNF, ILB) as predictor variables and microbial abundance data as a response. We found that IL17C rather than TNF or ILB had the strongest positive associations, comprising all major genera of the Enterobacteriaceae family (FIG. 6E; Table 3 and Table 5). The link between IL17C expression and relative abundance of Enterobacteriaceae in human mucosal biopsies supports the concept that analogous to Duoxa-deficient mice, high plasma IL17C levels are indicative of a shift in the gram-negative microbiota at the mucosal surface.

### Example II

This example links the detection of plasma IL17C to the risk of developing inflammatory bowel disease. It describes that variants in an epithelial host defense gene can be stratified based on their strength of association with plasma IL17C induction in non-IBD individuals. The results reveal that those variants associated with IL17C induction in non-IBD individuals confer a significant risk for the development of IBD.

### Methods

### Collection of Human Blood Samples.

The study was reviewed and approved by the Western IRB (Study Number 1178906). The research was performed entirely using de-identified and aggregated data of individuals who had signed a research authorization allowing the use of their anonymized data in research. Trained phlebotomists collected blood used for whole-genome sequencing, clinical laboratory tests, proteomics, and metabolomics in standard clinical facilities. Four days in advance of each blood draw, study participants were asked to discontinue non-prescription medications, including acetaminophen, ibuprofen, and over-the-counter cold remedies. 24 hours in advance of each blood draw, participants were asked to avoid alcohol, vigorous exercise, and products containing aspartame or MSG. 12 hours in advance of each blood draw, participants were asked to fast (no food or drink except water) until after the draw was completed. Non-fasting samples were excluded from this study.

### Whole-Genome Sequencing and DUOX2/DUOXA2 Variants Annotation.

DNA was extracted from whole blood samples for whole-genome sequencing in a CLIA-approved lab (Wuxi, Shanghai, China) using Illumina HiSeq X technology with sequencing mode PE150 and 30X target coverage. The sequenced reads were aligned to human reference GRCh37/hg19 using BWA 0.7.12. (28). Variant calling was performed with GATK 3.3.0, including indel local realignment followed by base quality recalibration (29). Variant calls were produced by GATK HaplotypeCaller. Only calls with DP>8 and GQ>20 were included in this study. The Ensembl GRCh37 annotation v75 was used to identify gene boundaries for DUOX2/DUOXA2. Variants passing quality filters were selected within these gene boundaries using custom Python scripts. The Ensembl Variant Effect Predictor REST API was used to assign the functional impact of each variant. The API query was defined as http://grch37.rest.ensembl.org/vep/human/region/{chr}: {start}-{end}:1/{allele}?CADD=1&Conservation=1&ExAC=1. The most severe consequence at each position was used to filter the variants. Variants were selected for downstream analysis if VEP consequence was one of {‘missense_variant’, ‘frameshift_variant’, ‘splice_acceptor_variant’, ‘splice_donor_variant’, ‘stop_gained’}

### Clinical Laboratory Tests.

Blood samples were analyzed at either LabCorp (North Carolina, USA) or Q² Solutions (North Carolina, USA). Clinical blood tests included diabetes markers, a lipid panel, complete blood cell counts, inflammation markers, liver function markers, kidney function markers, nutrition markers, and other markers, all of which were tested according to standard clinical procedures defined by the testing laboratories.

### Plasma Proteomics.

Plasma concentrations of proteins were measured using the ProSeek Cardiovascular II, Cardiovascular III, and Inflammation panels (Olink Biosciences, Uppsala, Sweden) at Olink facilities in Boston, MA. The ProSeek method is based on the highly sensitive and specific proximity extension assay, which involves the binding of distinct polyclonal oligonucleotide-labeled antibodies to the target protein followed by quantification with real-time quantitative polymerase chain reaction (rt-PCR) (30). Samples were processed in several batches; potential batch effects were adjusted using pooled control samples included with each batch.

### Plasma Metabolomics.

Metabolon Inc. (Durham, NC) conducted the metabolomics assays on plasma samples. Data were generated using the Global Discovery platform. Samples were processed in several batches with pooled quality control samples included in each batch; potential batch effects for each metabolite were adjusted by dividing by the corresponding average value identified in the pooled quality control samples from the same batch.

### Human Fecal Microbiome.

Individuals collected stool samples at home using the DNA Genotek OMNIGene GUT collection kit and shipped at ambient temperature to the sequencing laboratory. Baseline gut microbiome sequencing data in the form of FASTQ files were provided by Second Genome (California, USA) or DNA Genotek (Ottawa, Canada) based on 250 bp paired-end MiSeq profiling of the 16S v4 region. OTU abundances were calculated using the QIIME (31) pipeline and Greengenes database. PICRUSt (32) was used to infer metagenome functional content, and KEGG orthologies were collapsed into KEGG Pathways and KEGG Modules for analysis.

### Phenome-Wide Association Study.

Prior to performing the analyses, the highest and lowest 0.25% of values were winsorized. Highly skewed distributions (|skew|>1.5) were log-transformed prior to analysis. To adjust for potential confounding effects, the non-time-varying covariates age, sex, body mass index, enrollment channel, whether or not the participant reported taking cholesterol medications, blood pressure medications, or diabetes medications, and genetic ancestry, as well as the time-varying covariates observation month and observation vendor (when multiple vendors were used) were included as fixed effects in all models. Genetic ancestry was represented by principal components (PCs) 1-8 from an analysis of 107,280 linkage disequilibrium pruned autosomal SNPs with minor allele frequency >5% using the combined PC-AiR (33) and PC-Relate (34) approach as described by Conomos et al. (35). The GENESIS R package was used to perform SKAT-O tests using Madsen-Browning weights (36). Gaussian null models were used with test type Score.

### Site-Directed Mutagenesis and Heterologous Expression of DUOX2 Variants.

Individual DUOX2 variants were introduced into an N-terminal hemagglutinin epitope (HA)-tagged DUOX2 expression vector (37) by site-directed mutagenesis (QuikChange; Stratagene, La Jolla, CA). All constructs were verified by bidirectional Sanger sequencing (Supplementary Figure S2A). The DUOXA2-EGFP expression vector was prepared as described (37). HEK293 cells were transfected at 50-60% confluence using FuGENE 6 reagent (Promega, Madison, WI, USA). DUOXA2-EGFP (controls: EGFP and empty vector) was cotransfected with an equal amount (105 ng/cm² cell monolayer) of one of the DUOX2 plasmids (wildtype or variant, control: empty vector). Under these conditions, DUOXA2 is available in significant excess and does not limit DUOX2/DUOXA2 heterodimerization (38). In all experiments, the total amount of DNA in each transfection was kept constant by adjusting with the empty vector.

### DUOX2 Enzymatic Activity Assay.

H₂O₂ released into the culture medium was measured using a peroxidase-independent homogenous bioluminescence detection system (ROS Glo H₂O₂; Promega). Briefly, cells were washed and incubated at 37° C. for 1 h in HBSS(Ca²⁺)/10 mM HEPES (pH 7.4)/10 mM glucose containing 1 μM ionomycin/200 nM 12-O-tetradecanoylphorbol-13-acetate (TPA) to stimulate DUOX2 intrinsic activity and 25 ?M ROS-Glo Substrate that reacts with H₂O₂ to generate a luciferin precursor. Following incubation, aliquots of the culture medium were mixed with equal amounts of ROS-Glo Detection Solution containing recombinant luciferase, and luminescence was measured on a Synergy 2 plate reader (BioTek Instruments, Inc.). As an internal control for transfection efficiency, luciferase activity from cotransfected pGL3-Promoter (Promega) was determined in the remaining cells (Luciferase Assay; Biotium).

### Quantitation of DUOX2 Expression in the Plasma Membrane.

The flow cytometry assay to quantitate recombinant DUOX2 expression at the cell surface has been previously described in detail (4) (see Supplementary Figures S2B and S2C). Briefly, exposure of the N-terminal HA epitope of HA-DUOX2 in non-permeabilized cells was detected using rat anti-HA (clone 3F10, Roche) as primary and Alexa Fluor 647-conjugated anti-rat IgG as the secondary antibody, respectively. The intracellular EGFP moiety of the co-transfected DUOXA2-EGFP was used to select the population of transfected cells. Cytometry data were acquired on an Accuri C6 flow cytometer (BD Biosciences) (FL1: EGFP; FL4: AF647 nm) and analyzed using FlowJo v10.5.3 software. Relative DUOX2 surface expression (AUC of FL4 in EGFP⁺ cells) was normalized for the number of EGFP⁺ cells.

### The Burden of High Impact DUOX2 Mutations in IBD.

DUOX2 variant frequency data for IBD (4970 Non-Finnish European, 2641 Ashkenazi Jewish, 696 Finnish) and control cohorts (2770 Non-Finnish European, 3044 Ashkenazi Jewish, 9930 Finnish) were obtained from the IBD Exomes Portal, Cambridge, MA (URL: http://ibd.broadinstitute.org). Genotype quality control and relatedness filter have been described (39). Protein-altering variants were selected using Ensembl VEP classifier. Ancestry-specific minor allele frequencies for stratification were obtained from gnomAD v2.1. Odds ratios (OR) were calculated from cumulative allele frequency data. The combined effect size for all cohorts was estimated using a random effect model with Mantel-Haenszel weighting (16). The point estimate for the proportion of observed variance in OR between cohorts that reflects real OR differences (I²) was 36%.

### Results

1) Variants in an Epithelial Host Defense Gene are Associated with Outlier High Plasma IL17C Concentration in the General Population.

To better understand the role of IL17C induction as predictor of abnormal gut microbe-immune homeostasis and the risk for developing inflammatory disease, we examined the effect of genetic variants in the DUOX2 heterodimer NADPH oxidase (subunits: DUOX2 and DUOXA2) as paradigm for disturbed immune homeostasis. DUOX2 is an evolutionary conserved host defense system responsible for microbial-induced hydrogen peroxide (H₂O₂) release at the apical surface of gut epithelial cells. By its function and regulation, DUOX2 has been considered a candidate susceptibility factor for IBD.

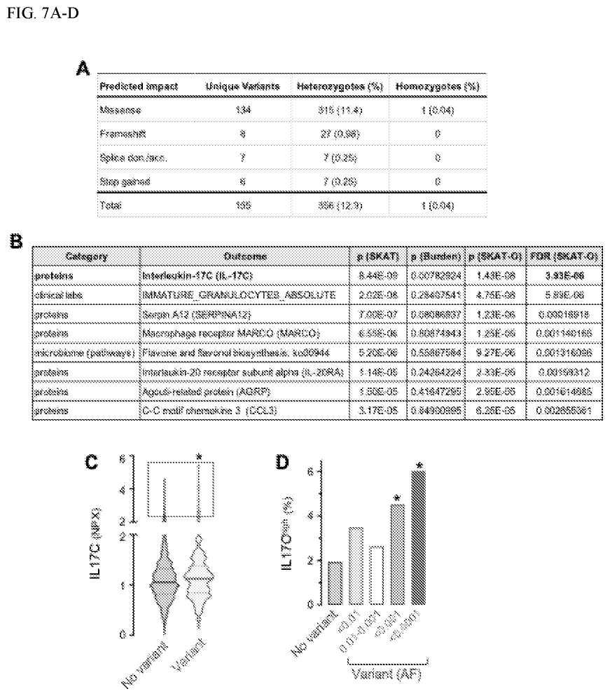

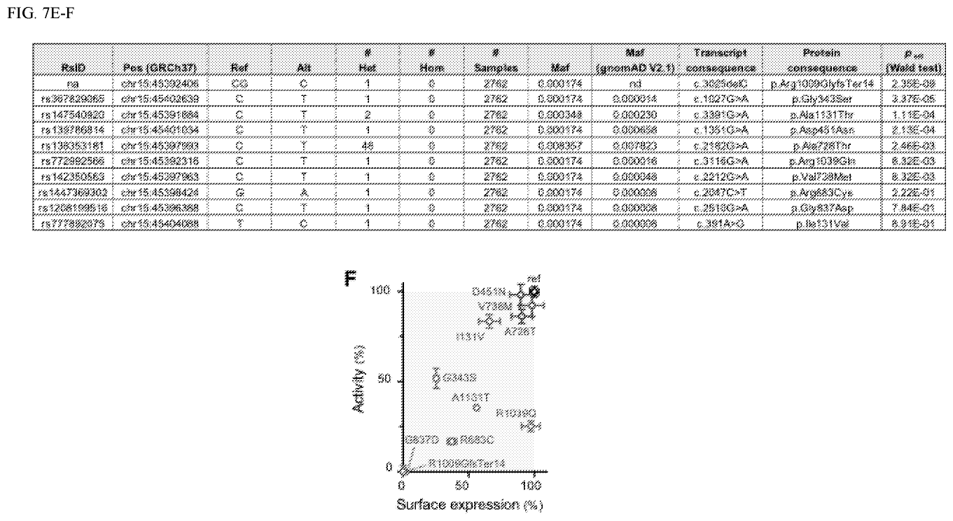

We previously noted a substantial burden of rare protein-altering DUOX2 variants of unknown significance in the general population (40). For an unbiased exploration of the phenotypic impact of such variants, we carried out a multi-omic phenome-wide association study (PheWAS) with data from 2,762 participants in a commercial lifestyle coaching program (Arivale). Genetic variants falling within the DUOX2 and DUOXA2 (essential DUOX2 heterodimerization partner) exonic boundaries and passing quality filters were annotated with the Ensembl Variant Effect Predictor; only protein-altering variants were included in downstream analyses. In total, we identified 155 unique alleles with <1% frequency each (FIG. 7A). Of the 357 (12.9%) individuals with rare variants, a large majority (328) carried only a single heterozygous variant.

We used optimal unified sequence kernel association (SKAT-O) tests to find statistical associations between the identified variants and quantitative phenotypes comprising 124 clinical laboratory tests, 951 plasma metabolites, 266 plasma proteins, and 16S rRNA-based profiling data of the fecal microbiome. We found that protein-altering DUOX2/DUOXA2 variants were most significantly associated with the plasma level of interleukin-17C (IL17C; FDR=2.6e−5) (FIG. 7B). The distributions of IL17C values differed between variant-carriers and individuals without variant (p=0.042) with the former having a more right-tail heavy distribution (positive skewness 2.63 vs 1.65; kurtosis 13.79 vs 6.85) (FIG. 7C). For further analysis, we stratified carriers based on minor allele rarity, a strong predictor of deleteriousness (41). The prevalence of abnormally high plasma IL17C levels indeed substantially increased with allele rarity in ancestry-matched control populations (FIG. 7D). To formally assess IL17C-associated DUOX2 variants for their impact on the enzyme's activity, we tested ten variants with the most significant contribution to the association signal (FIG. 7E) in a heterologous expression system (4). We confirmed a significant functional impairment for the majority of tested alleles (FIG. 7F). Thus, partially impaired DUOX2 function due to rare protein-altering variants is a frequent finding in the general population; carriers of such variants are prone to have excessively high plasma IL17C levels.

2) Variants in an Epithelial Host Defense Gene that are Associated with Plasma IL17C Induction in Non-IBD Subjects Confer Increased Risk for Developing IBD.

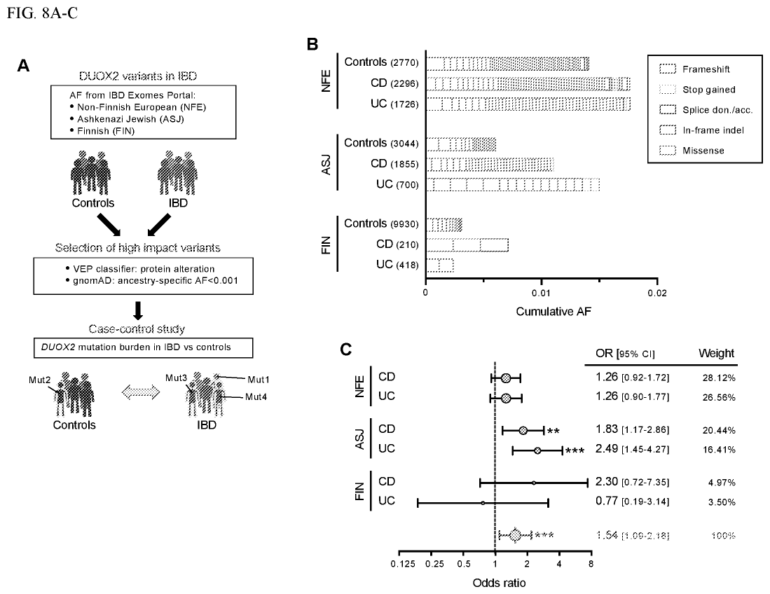

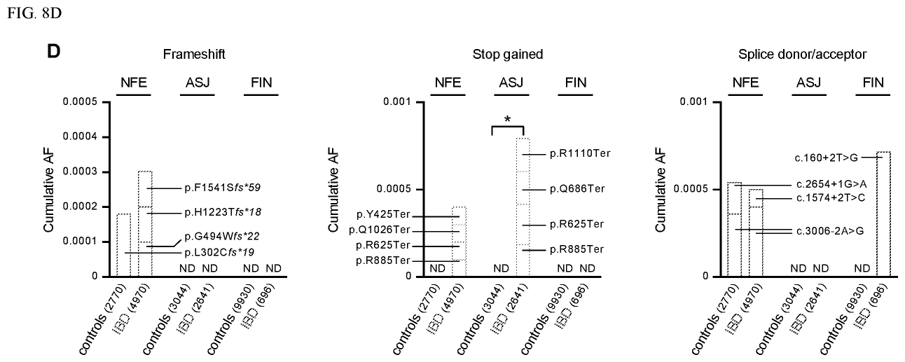

To directly assess whether abnormally high plasma IL17C levels found in the context of rare DUOX2 protein variants correlate with increased risk for developing IBD, variants detected in whole genome-sequencing data of large IBD cohorts (IBD Exomes Portal) were classified as high impact variants using the same criteria for which we observed a significantly increased prevalence of outlier high plasma IL17C concentrations in the PheWAS cohort (FIGS. 8A and 8B). Using a meta-analysis of the three IBD cohorts, we found a significantly increased risk among DUOX2 variant carriers to develop IBD (pooled odds ratio (OR)=1.54 [95% CI:1.09-2.18]; p=0.0007; random-effects model) (FIG. 8C). With respect to the specific ancestry groups, there was a significant effect of DUOX2 variants on IBD risk in the Ashkenazi Jewish (ASJ) cohort with an OR estimate of 2.13 (95% CI: 1.427-3.187; p=0.0002; 2-tailed Yates's chi-squared test). For the Non-Finnish European cohorts, the calculated OR for IBD was 1.27, but the result did not pass the significance threshold (95% CI 0.977-1.67; p=0.0741; 2-tailed Yates's chi-squared test). Note that for the Finnish cohort, the smaller size of this IBD cohort and genetic bottlenecks in this population leading to a lower rate of very rare variants severely limited the statistical power of this analysis (IBD: OR=1.3823 [0.5969-3.2013]; p=0.4498). Concerning IBD subtypes, the risk associated with DUOX2 variants appeared to be similar for CD and UC patients (FIG. 8C). To check for internal consistency of these associations we reviewed the small subset of predicted null variants (i.e., nonsense, frameshift, canonical splice donor, or acceptor site mutations) that should each confer the maximum possible risk for heterozygous DUOX2 variants (FIG. 8D). Compared to the overall high impact variant selection, the distribution of null variants was indeed suggestive of even more pronounced enrichment among IBD patients. Thus, high plasma IL17C in carriers of DUOX2 loss-of-function variants is not only a potential biomarker for disturbed gut microbe-immune homeostasis but appears to reflect an early stage of IBD pathogenesis.

### Example III

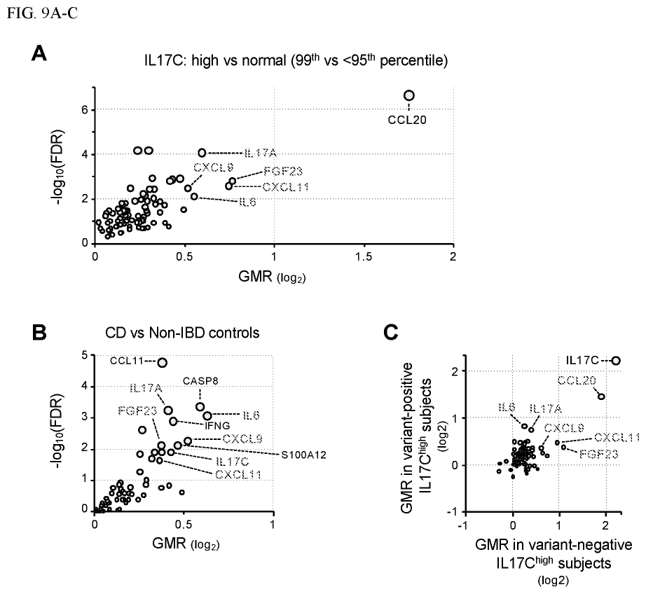

This example illustrates additional serological markers that can be combined with the IL17C assay into a biomarker panel to identify subjects with proinflammatory mucosal dysbiosis that are candidates for preventive therapeutic measures aiming to restore immune homeostasis. In our study population (without IBD diagnosis), the IL17C^{high} phenotype was associated with frequent elevation of specific other inflammation-related plasma proteins. Of these, the chemokine CCL20, the unique ligand for CCR6-mediated recruitment of Th17 cells, was most consistently increased in concert with high IL17C. In the healthy gut, CCL20 shows only weak constitutive expression in the surface epithelial layer, predominantly the follicle-associated epithelium in the small intestine. Bacterial contact triggers CCL20 expression either directly via toll-like receptor-dependent signaling (42) or indirectly by being an IL17C downstream target (43). Apart from CCL20, IL17C^{high} subjects had significantly higher mean plasma levels of CXCL9, CXCL11, FGF23, IL6, and IL17A (FIG. 9A). With respect to the latter proteins, it is noteworthy that they belong to a plasma protein signature that is commonly upregulated in the plasma of CD patients (7) (FIG. 9B). The presence of “IBD biomarkers” in IL17C^{high} subjects was not driven by the inclusion of individuals with self-reported IBD diagnosis. Thus, in IL17C^{high} subjects without prior IBD diagnosis, the plasma protein profile is frequently compatible with a concerted gut mucosal immune response (microinflammation). This specific chemokine/cytokine signature was not unique to carriers of DUOX2 variants, but similarly found in IL7C^{high} subjects without DUOX2 variant (FIG. 9C)

### Example IV

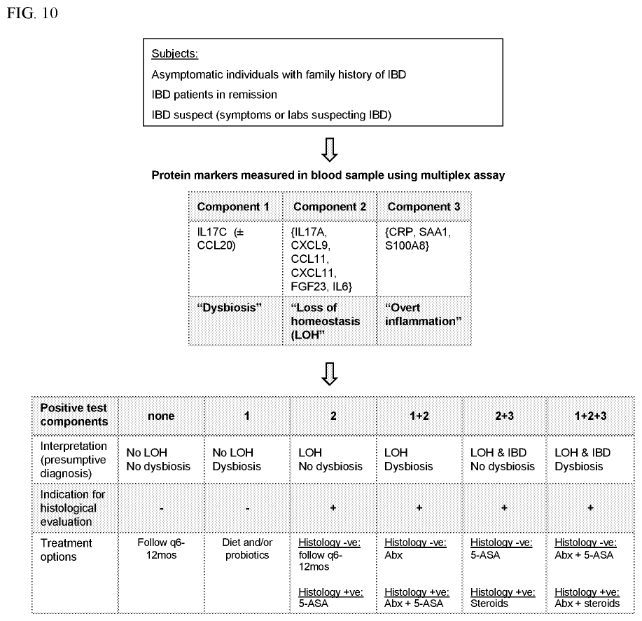

This example illustrates how the results from a multiplex biomarker test kit can be integrated into a diagnostic and treatment algorithm (FIG. 10). The test kit evaluates the blood level of IL17C (with or without CCL20) as a marker for abnormal activation of the gut epithelium by components of the microbiota (condition 1: mucosal dysbiosis), a profile of proteins indicating loss of immune homeostasis (condition 2: LOH), and markers indicating severe inflammation (condition 3: overt inflammation). Patients are stratified based on the results of the individual test components. A test result consistent with LOH guides the decision to pursue additional colonoscopy and biopsies (e.g., presence of micro or macroinflammation). The treatment algorithm guides the selection of the most appropriate treatment modalities. It is based on the classification obtained using the results from the test kit and histological findings of inflammation in endoscopic biopsies if indicated. For instance, the presence of condition 1 (mucosal dysbiosis) but not of condition 2 (LOH) indicates increased contact of gut microbes with the mucosal surface that has not initiated a (pro-) inflammatory process. These patients are at increased risk for developing microbiota-driven inflammatory disease. Conservative microbiota-modulating therapies consisting of dietary intervention and/or probiotics can be considered on a case-by-case basis. Normalization of IL17C level during or following treatment is indicative of a reduction in abnormal microbiota-epithelial interactions. Presence of conditions 1 and 2 (dysbiosis with evidence for loss of homeostasis) indicates that a (pro-)inflammatory process in the mucosa is driven by the microbiota. These patients are prime candidates for antibiotics treatment that is combined with anti-inflammatory treatment (5-ASA) if histological inflammation is present. Presence of condition 2 (LOH) without condition 1 (dysbiosis) indicates that the (pro-)inflammatory process in the mucosa is not currently driven by abnormal interaction with the gut microbiota. In these patients, anti-inflammatory treatment such as 5-ASA is indicated for cases with confirmed gut microinflammation, but unnecessary and potentially harmful treatment with antibiotics is to be avoided.

Having now fully described the invention, it will be understood by those of skill in the art that the same can be performed within a wide and equivalent range of conditions, formulations, and other parameters without affecting the scope of the invention or any embodiment thereof. All patents, patent applications, and publications cited herein are fully incorporated by reference herein in their entirety.

### INCORPORATION BY REFERENCE

The entire disclosure of each of the patent documents and scientific articles referred to herein is incorporated by reference for all purposes. Each of the following references, numerically referred to herein, are herein incorporated by reference for all purposes:
1. Segata N, Izard J, Waldron L, Gevers D, Miropolsky L, Garrett W S, et al. Metagenomic biomarker discovery and explanation. Genome Biol. 2011;12(6):R60.
2. Chassaing B, Koren O, Goodrich J K, Poole A C, Srinivasan S, Ley R E, et al. Dietary emulsifiers impact the mouse gut microbiota promoting colitis and metabolic syndrome. Nature. 2015; 519(7541):92-6.
3. Jourquin J, Duncan D, Shi Z, and Zhang B. GLAD4U: deriving and prioritizing gene lists from PubMed literature. BMC Genomics. 2012;13 Suppl 8:S20.
4. Levine A P, Pontikos N, Schiff E R, Jostins L, Speed D, Consortium NIBDG, et al. Genetic Complexity of Crohn's Disease in Two Large Ashkenazi Jewish Families. Gastroenterology. 2016; 151(4):698-709.
5. Chheda H, Palta P, Pirinen M, McCarthy S, Walter K, Koskinen S, et al. Whole-genome view of the consequences of a population bottleneck using 2926 genome sequences from Finland and United Kingdom. Eur J Hum Genet. 2017; 25(4):477-84.
6. Sheskin D J. In: Sheskin D J ed. Handbook of parametric and nonparametric statistical procedures. Boca Raton: Chapman & Hall/CRC; 2007.
7. Andersson E, Bergemalm D, Kruse R, Neumann G, D'Amato M, Repsilber D, et al. Subphenotypes of inflammatory bowel disease are characterized by specific serum protein profiles. PLoS One. 2017;12(10):e0186142.
8. Fernando E H, Dicay M, Stahl M, Gordon M H, Vegso A, Baggio C, et al. A simple, cost-effective method for generating murine colonic 3D enteroids and 2D monolayers for studies of primary epithelial cell function. Am J Physiol Gastrointest Liver Physiol. 2017;313(5):G467-G75.
9. Buffie C G, Bucci V, Stein R R, McKenney P T, Ling L, Goboume A, et al. Precision microbiome reconstitution restores bile acid mediated resistance to Clostridium difficile. Nature. 2015; 517(7533):205-8.
10. Grasberger H, El-Zaatari M, and Merchant J L. Dual Oxidases Control Release of Hydrogen Peroxide by the Gastric Epithelium to Prevent Helicobacter felis Infection and Inflammation in Mice. Gastroenterology. 2013.
11. Grasberger H, De Deken X, Mayo O B, Raad H, Weiss M, Liao X H, et al. Mice deficient in dual oxidase maturation factors are severely hypothyroid. Mol Endocrinol. 2012; 26(3):481-92.
12. Umesaki Y, Okada Y, Matsumoto S, Imaoka A, and Setoyama H. Segmented filamentous bacteria are indigenous intestinal bacteria that activate intraepithelial lymphocytes and induce MHC class II molecules and fucosyl asialo GM1 glycolipids on the small intestinal epithelial cells in the ex-germ-free mouse. Microbiol Immunol. 1995; 39(8):555-62.
13. Schloss P D, Westcott S L, Ryabin T, Hall J R, Hartmann M, Hollister E B, et al. Introducing mothur: open-source, platform-independent, community-supported software for describing and comparing microbial communities. Appl Environ Microbiol. 2009; 75(23):7537-41.
14. Weber N, Liou D, Dommer J, MacMenamin P, Quinones M, Misner I, et al. Nephele: a cloud platform for simplified, standardized and reproducible microbiome data analysis. Bioinformatics. 2018; 34(8):1411-3.
15. Morgan X C, Tickle T L, Sokol H, Gevers D, Devaney K L, Ward D V, et al. Dysfunction of the intestinal microbiome in inflammatory bowel disease and treatment. Genome Biol. 2012;13(9):R79.
16. Suurmond R, van Rhee H, and Hak T. Introduction, comparison, and validation of Meta-Essentials: A free and simple tool for meta-analysis. Res Synth Methods. 2017; 8(4):537-53.
17. Wang J, Vasaikar S, Shi Z, Greer M, and Zhang B. WebGestalt 2017: a more comprehensive, powerful, flexible and interactive gene set enrichment analysis toolkit. Nucleic Acids Res. 2017;45(W1):W130-W7.
18. Grasberger H, Gao J, Nagao-Kitamoto H, Kitamoto S, Zhang M, Kamada N, et al. Increased Expression of DUOX2 Is an Epithelial Response to Mucosal Dysbiosis Required for Immune Homeostasis in Mouse Intestine. Gastroenterology. 2015; 149(7):1849-59.
19. Ivanov, II, Atarashi K, Manel N, Brodie E L, Shima T, Karaoz U, et al. Induction of intestinal Th17 cells by segmented filamentous bacteria. Cell. 2009; 139(3):485-98.
20. Larsson E, Tremaroli V, Lee Y S, Koren O, Nookaew I, Fricker A, et al. Analysis of gut microbial regulation of host gene expression along the length of the gut and regulation of gut microbial ecology through MyD88. Gut. 2012; 61(8):1124-31.
21. Im E, Jung J, and Rhee S H. Toll-like receptor 5 engagement induces interleukin-17C expression in intestinal epithelial cells. J Interferon Cytokine Res. 2012; 32(12):583-91.
22. Pircalabioru G, Aviello G, Kubica M, Zhdanov A, Paclet M H, Brennan L, et al. Defensive Mutualism Rescues NADPH Oxidase Inactivation in Gut Infection. Cell Host Microbe. 2016; 19(5):651-63.
23. Collins K D, Hu S, Grasberger H, Kao J Y, and Ottemann K M. Chemotaxis Allows Bacteria To Overcome Host-Generated Reactive Oxygen Species That Constrain Gland Colonization. Infect Immun. 2018;86(5).
24. Johansson M E, Larsson J M, and Hansson G C. The two mucus layers of colon are organized by the MUC2 mucin, whereas the outer layer is a legislator of host-microbial interactions. Proc Natl Acad Sci USA. 2011;108 Suppl 1:4659-65.
25. Haberman Y, Tickle T L, Dexheimer P J, Kim M O, Tang D, Kams R, et al. Pediatric Crohn disease patients exhibit specific ileal transcriptome and microbiome signature. J Clin Invest. 2014; 124(8):3617-33.
26. Gevers D, Kugathasan S, Denson L A, Vazquez-Baeza Y, Van Treuren W, Ren B, et al. The treatment-naive microbiome in new-onset Crohn's disease. Cell Host Microbe. 2014; 15(3):382-92.
27. Winter S E, Lopez C A, and Baumler A J. The dynamics of gut-associated microbial communities during inflammation. EMBO Rep. 2013; 14(4):319-27.
28. Li H, and Durbin R. Fast and accurate short read alignment with Burrows-Wheeler transform. Bioinformatics. 2009; 25(14):1754-60.
29. McKenna A, Hanna M, Banks E, Sivachenko A, Cibulskis K, Kernytsky A, et al. The Genome Analysis Toolkit: a MapReduce framework for analyzing next-generation DNA sequencing data. Genome Res. 2010; 20(9):1297-303.
30. Lundberg M, Eriksson A, Tran B, Assarsson E, and Fredriksson S. Homogeneous antibody-based proximity extension assays provide sensitive and specific detection of low-abundant proteins in human blood. Nucleic Acids Res. 2011;39(15):e102.
31. Caporaso J G, Kuczynski J, Stombaugh J, Bittinger K, Bushman F D, Costello E K, et al. QIIME allows analysis of high-throughput community sequencing data. Nat Methods. 2010; 7(5):335-6.
32. Langille M G, Zaneveld J, Caporaso J G, McDonald D, Knights D, Reyes J A, et al. Predictive functional profiling of microbial communities using 16S rRNA marker gene sequences. Nat Biotechnol. 2013; 31(9):814-21.
33. Conomos M P, Miller M B, and Thornton T A. Robust inference of population structure for ancestry prediction and correction of stratification in the presence of relatedness. Genet Epidemiol. 2015; 39(4):276-93.
34. Conomos M P, Reiner A P, Weir B S, and Thornton T A. Model-free Estimation of Recent Genetic Relatedness. Am J Hum Genet. 2016; 98(1):127-48.
35. Conomos M P, Laurie C A, Stilp A M, Gogarten S M, McHugh C P, Nelson S C, et al. Genetic Diversity and Association Studies in US Hispanic/Latino Populations: Applications in the Hispanic Community Health Study/Study of Latinos. Am J Hum Genet. 2016; 98(1):165-84.
36. Madsen B E, and Browning S R. A groupwise association test for rare mutations using a weighted sum statistic. PLoS Genet. 2009;5(2):e1000384.
37. Grasberger H, and Refetoff S. Identification of the maturation factor for dual oxidase. Evolution of an eukaryotic operon equivalent. J Biol Chem. 2006; 281(27):18269-72.
38. Grasberger H, De Deken X, Miot F, Pohlenz J, and Refetoff S. Missense mutations of dual oxidase 2 (DUOX2) implicated in congenital hypothyroidism have impaired trafficking in cells reconstituted with DUOX2 maturation factor. Mol Endocrinol. 2007; 21(6):1408-21.
39. Rivas M A, Avila B E, Koskela J, Huang H, Stevens C, Pirinen M, et al. Insights into the genetic epidemiology of Crohn's and rare diseases in the Ashkenazi Jewish population. PLoS Genet. 2018;14(5):e1007329.
40. Grasberger H, Noureldin M, Kao T D, Adler J, Lee J M, Bishu S, et al. Increased risk for inflammatory bowel disease in congenital hypothyroidism supports the existence of a shared susceptibility factor. Sci Rep. 2018;8(1):10158.
41. Kryukov G V, Pennacchio L A, and Sunyaev S R. Most rare missense alleles are deleterious in humans: implications for complex disease and association studies. Am J Hum Genet. 2007; 80(4):727-39.
42. Skovdahl H K, Granlund A, Ostvik A E, Bruland T, Bakke I, Torp S H, et al. Expression of CCL20 and Its Corresponding Receptor CCR6 Is Enhanced in Active Inflammatory Bowel Disease, and TLR3 Mediates CCL20 Expression in Colonic Epithelial Cells. PLoS One. 2015;10(11):e0141710.
43. Ramirez-Carrozzi V, Sambandam A, Luis E, Lin Z, Jeet S, Lesch J, et al. IL-17C regulates the innate immune function of epithelial cells in an autocrine manner. Nat Immunol. 2011; 12(12):1159-66.

What is claimed is:

1. A method, comprising:
(a) measuring an IL17C level in a biological sample obtained from a subject;
(b) characterizing the measured IL17C level within an established IL17C range;
(c) measuring the levels of one or more of interleukin 17A (IL17A), interleukin 6 (IL6), C—C motif chemokine ligand 20 (CCL20), C—X—C motif chemokine ligand 9 (CXCL9), C—C Motif Chemokine Ligand 11 (CCL11), C—X—C motif chemokine ligand 11 (CXCL11), Fibroblast growth factor-23 (FGF23), and one or more of C-reactive protein (CRP), serum amyloid A (SAA1), and neutrophilic marker calprotectin (S100A8), within the biological sample if the measured IL17C level is characterized as elevated within the established IL17C range;
(d) characterizing the one or more measured IL17A, IL6, CCL20, CXCL9, CCL11, CXCL11, FGF23, CRP, and S100A8 levels within established ranges for IL17C, IL17A, IL6, CCL20, CXCL9, CCL11, CXCL11, FGF23, CRP, SAA1, and S100A8 levels;
(e) characterizing an intestinal inflammation status for the subject based upon the one or more characterized IL17A, IL6, CCL20, CXCL9, CCL11, CXCL11, FGF23, CRP, SAA1, and S100A8 levels; and
(f) treating the characterized intestinal inflammation status in the subject.

2. The method of claim 1, where the subject is a human subject suffering or at risk of suffering from a breakdown of microbiota/immune system homeostasis.

3. The method of claim 1, where the subject is a human subject suffering or at risk of suffering from an expansion of proteobacteria pathobionts.

4. The method of claim 1, where the subject is a human subject suffering or at risk of suffering from inflammatory bowel disease (IBD) due to a loss of microbiota/immune system homeostasis at gut epithelial surfaces.

5. The method of claim 1, where the subject is a human subject who has IBD, is diagnosed with IBD, is suspected to have IBD, is likely to have IBD, has one or more signs or symptoms of IBD (e.g., gastrointestinal, systemic, and extraintestinal symptoms), has increased risk for developing IBD based on positive family history or the presence of one or more risk variants in IBD susceptibility genes.

6. The method of claim 1, where the subject is a human subject has been previously diagnosed with irritable bowel syndrome (IBS), obesity, metabolic syndrome, hepatic encephalopathy, colon cancer.

7. The method of claim 1, wherein the biological sample is a blood sample (e.g., plasma, serum, whole blood).

8. The method of claim 1, wherein the biological sample is a tissue sample (e.g., an intestinal tissue sample).

9. The method of claim 1, wherein the established marker (IL17C, IL17A, IL6, CCL20, CXCL9, CCL11, CXCL11, FGF23, CRP, SAA1, and S100A8 levels) range is an established range of levels for that specific marker generated from a plurality of subjects (e.g., human subjects) (e.g., human subjects not suffering from intestinal inflammation and human subjects suffering from intestinal inflammation).

10. The method of claim 1, wherein a measured IL17C level characterized as elevated is within the top 10% of the established 1L17C level range.

11. The method of claim 1, wherein a measured IL17C level characterized as elevated is within the top 5% of the established 1L17C level range.

12. The method of claim 1, wherein a measured IL17C level characterized as elevated is within the top 2% of the established 1L17C level range.

13. The method of claim 1, wherein a measured IL17C level characterized as elevated is within the top 1% of the established 1L17C level range.

14. The method of claim 1, wherein the subject is characterized as not having intestinal mucosal dysbiosis if the measured levels of IL17C is characterized as not elevated in comparison with the established IL17C level.

15. The method of claim 1, wherein the subject is characterized as having mucosal dysbiosis without loss of homeostasis if the measured level of IL17C is characterized as elevated within the established IL17C level range, and each of IL17A, IL6, CXCL9, CCL11, CXCL11, FGF23, CRP, SAA1, and S100A8 is characterized as not elevated within the established range of levels for each specific marker.

16. The method of claim 1, wherein the subject is characterized as having mucosal dysbiosis with loss of homeostasis (microinflammation) if the measured level of IL17C is characterized as elevated within the established IL17C level range, and one or more of IL17A, IL6, CXCL9, CCL11, CXCL11, and FGF23 is characterized as elevated within the established range of levels for each specific marker.

17. The method of claim 1, wherein the subject is characterized as having mucosal dysbiosis in the context of overt inflammation if the measured level of IL17C is characterized as elevated within the established IL17C level range, and one or more of CRP, SAA1, and S100A8 is characterized as elevated within the established range of levels for each specific marker.

18. The method of claim 15, wherein the subject is treated through administration of a therapeutically effective amount of one or more agents selected from a prebiotic agent, a probiotic agent, and a postbiotic agent.

19. The method of claim 18, wherein the prebiotic agent is selected from the group consisting of: complex carbohydrates, complex sugars, resistant dextrins, resistant starch, amino acids, peptides, nutritional compounds, biotin, polydextrose, fructooligosaccharide (FOS), galactooligosaccharides (GOS), inulin, starch, lignin, psyllium, chitin, chitosan, gums (e.g. guar gum), high amylose cornstarch (HAS), cellulose, beta-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, xylooligosaccharides (XOS), locust bean gum, beta-glucan, methylcellulose, and any combination thereof.

20. The method of claim 18, wherein the prebiotic agent is an oligosaccharide.

21. The method of claim 18, wherein the prebiotic agent is inulin.

22. The method of claim 18, wherein the prebiotic agent is selected from the group consisting of: amino acids, ammonium nitrate, amylose, barley mulch, biotin, carbonate, cellulose, chitin, choline, fructooligosaccharides (FOSs), fructose, galactooligosaccharides (GOSs), glucose, glycerol, heteropolysaccharide, histidine, homopolysaccharide, hydroxyapatite, inulin, isomaltulose, lactose, lactulose, maltodextrins, maltose, mannooligosaccharides, tagatose, nitrogen, oligodextrose, oligofructoses, oligofructose-enriched inulin, oligosaccharides, pectin, phosphate salts, phosphorus, polydextroses, polyols, potash, potassium, sodium nitrate, starch, sucrose, sulfur, sun fiber, tagatose, thiamine, trans-galactooligosaccharides, trehalose, vitamins, a water-soluble carbohydrate, and/or xylooligosaccharides (XOSs).

23. The method of claims 16 or 17, wherein the subject is treated through administration of a therapeutically effective amount of one or more antibiotic agents.

24. The method of claim 23, wherein the antibiotic is selected from the group consisting of: rifabutin, clarithromycin, clofazimine, vancomycin, rifampicin, nitroimidazole, chloramphenicol, and a combination thereof. In another aspect, an antibiotic composition administered herein comprises an antibiotic selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, and a combination thereof.

25. A kit comprising one or more of a prebiotic agent, a probiotic agent, a postbiotic agent, an antibiotic, and reagents capable of measuring one or more of IL17C, IL17A, IL6, CCL20, CXCL9, CCL11, CXCL11, FGF23, CRP, SAA1, and S100A8 levels within a biological sample.

26. A method of treating gut dysbiosis in a subject, comprising:
(a) measuring the levels of first and second proteins in a blood and/or tissue sample of the subject, the first protein interleukin 17C (IL-17C) and the second protein depicting the intestinal inflammation status of the subject selected from (i) a biomarker of loss of gut epithelial homeostasis, (ii) a biomarker of overt gut epithelial inflammation, and (iii) combinations of (i) and (ii); and
(b) treating the subject for (i) micro-inflammatory gut dysbiosis when the subject is characterized as having elevated levels of the first and second proteins relative to an established range, the second protein a biomarker of a loss of gut epithelial homeostasis, or (ii) macro-inflammatory gut dysbiosis when the subject is characterized as having elevated levels of the first and second proteins relative to an established range, the second protein a biomarker of overt gut epithelial inflammation.

27. The method of claim 26, wherein the first protein further includes C—C motif chemokine ligand 20 (CCL20).

28. The method of claim 26, wherein the biomarker of loss of gut epithelial homeostasis is selected from IL17A, IL6, CXCL9, CCL11, CXCL11, and FGF23.

29. The method of claim 26, wherein the biomarker of overt gut epithelial inflammation is selected from CRP, SAA1, and S100A8.

30. The method of claim 26, wherein (a) gut epithelial homeostasis is characterized by normal levels of inflammation biomarker proteins interleukin IL17A, IL6, CXCL9, CCL11, CXCL11, FGF23, and (b) gut epithelial inflammation is characterized by normal levels of CRP, SAA1, and S100A8.

31. The method of claim 26, wherein the treatment for micro-inflammatory gut dysbiosis is selected from prebiotics, probiotics, and antibiotics.

32. The method of claim 26, wherein the treatment for macro-inflammatory gut dysbiosis is standard IBD treatment.

33. A kit comprising reagents capable of measuring levels within a biological sample of IL17C, one or more biomarkers of loss of gut epithelial homeostasis, and one or more biomarkers of overt gut epithelial inflammation.
